(19) United States
(12) Patent Application Publication
(10) Pub. No.: US 2022/0381777 A1
(43) Pub. Date: Dec. 1, 2022

(54) SOLUTION PHASE SINGLE MOLECULE CAPTURE AND ASSOCIATED TECHNIQUES

(71) Applicant: Palamedrix, Inc., La Jolla, CA (US)

(72) Inventors: Ashwin Gopinath, Palo Alto, CA (US); Paul Rothemund, Pasadena, CA (US); Rishabh Shetty, San Diego, CA (US); Shane Bowen, Encinitas, CA (US); Rachel Galimidi, Encinitas, CA (US); Dajun Yuan, San Diego, CA (US)

(21) Appl. No.: 17/825,867

(22) Filed: May 26, 2022

Related U.S. Application Data
(60) Provisional application No. 63/249,367, filed on Sep. 28, 2021.
(60) Provisional application No. 63/194,005, filed on May 27, 2021.

(51) Int. Cl. G01N 33/543; G01N 21/64

(52) U.S. Cl. CPC G01N 33/54387; G01N 33/54313; G01N 21/6428; G01N 2021/6439

(57) ABSTRACT

Provided herein are structures and methods for detecting one or more analyte molecules present in a sample. In some embodiments, the one or more analyte molecules form a complex in solution with a supramolecular structure. The supramolecular structures of the complex may be detectable such that binding of the analyte molecule to a binding site of an array is detectable via one or more features of the supramolecular structure. A binding site of an array includes capture molecules to capture bound complexes to facilitate detection.

### CROSS-REFERENCE TO RELATED APPLICATIONS

This application claims priority to U.S. Provisional Application 63/194,005, filed May 27, 2021 and to U.S. Provisional Application 63/249,367, filed Sep. 28, 2021, the disclosures of which are hereby incorporated by reference in their entireties herein for all purposes.

### BACKGROUND

The current state of personalized healthcare is overwhelmingly genome-centric, predominantly focused on quantifying the genes present within an individual. While such an approach has proven to be extremely powerful, it does not provide a clinician with the complete picture of an individual's health. This is because genes are the “blueprints” of an individual, and it merely informs the likelihood of developing an ailment. Within an individual, these “blueprints” first need to be transcribed into RNA and then translated into various protein molecules, “actors” in the cell, to have effect on the health of an individual.

The concentration of proteins, the interaction between the proteins (protein-protein interactions or PPI), as well as the interaction between proteins and other molecules, are intricately linked to the health of different organs, homeostatic regulatory mechanism as well as the interaction of these systems with the external environment. Hence, quantitative information about proteins and protein interactions such as PPIs is vital to create a complete picture of an individual's health at a given time point as well as to predict any emerging health issues. Presence and interactions between these proteins are also essential for drug development and are increasingly becoming a highly sought-after dataset to capture an individual proteome and changes to the proteome in response to environmental or other systemic events. The ability to detect and quantify proteins and protein interaction with other molecules within a given sample is an integral component of such healthcare development.

### SUMMARY

The present disclosure generally relates to systems, structures and methods for detection and quantification of analyte molecules in a sample.

Provided herein, in some embodiments, are solution-based techniques for detecting an analyte molecule present in a sample. The techniques include a sample preparation step in which analytes in solution are captured by or otherwise associated with respective supramolecular structures. For example, in an embodiment, an individual analyte (e.g., a protein molecule) is captured by an affinity binder of a supramolecular structure. Once captured, the analyte-supramolecular structure complex can be detected on substrate as part of a detection system, whereby individual binding sites of the substrate carry affinity binders for analytes of interest. Binding at a binding site of an analyte-supramolecular structure complex may be a sandwich-type arrangement in which the binding site affinity binder and the supramolecular structure affinity binder are bound to different locations on an individual analyte molecule. Thus, a detected signal at a particular binding site can be associated with the presence of a particular analyte of interest in the sample. The supramolecular structure of the complex can provide one or more of 1) a tag or barcode (e.g., a nucleic acid having a unique barcode sequence) to identify the associated affinity binder of an individual supramolecular structure, 2) a detectable initiator to provide a detectable signal that can be correlated to binding in the detection system, and 3) a physical scaffold coupled to the analyte that promotes single-molecule binding or low averages of molecules at a binding site of the substrate, e.g., that sterically hinders or sterically occludes binding of other molecules at the same binding site.

The size-excluding supramolecular structure is complexed with the analyte in solution, which creates greater flexibility in preparing a substrate for detection of the complexed analyte. In contrast to systems that are designed to have a single affinity binder associated with each binding site, which is complex to manufacture, the disclosed techniques work with more permissive binding site preparations that include a plurality of immobilized affinity binders at each binding site. Thus, the supramolecular structure can be a single-molecule binding entity, while the substrate can be arranged to permit multi-molecule binding at each binding site. The nature of the low number or single molecule binding at each binding site, facilitated by the supramolecular structure that space restricts binding at each site, permits high-throughput parallel characterization of multiple protein interactions on a single detection platform. Further, the solution-based sample preparation is more streamlined relative to substrate-based sample preparation.

As provided herein, a method for detecting an analyte molecule present in a sample can include providing a sample comprising analyte molecules and contacting the sample with a pool of supramolecular structures in solution to form analyte molecule-supramolecular structure complexes. The supramolecular structure can include a core structure comprising a plurality of core molecules and an affinity binder linked to the core structure. An individual analyte molecule-supramolecular structure complex can include a core structure comprising a plurality of core molecules; an affinity binder linked to the core structure; and an analyte molecule of the analyte molecules bound to the affinity binder, wherein different supramolecular structures of the pool of supramolecular structures comprise different affinity binders with different binding affinity for other analyte molecules of the analyte molecules. The method also includes contacting the analyte molecule-supramolecular structure complexes with an array, wherein binding sites of the array comprise respective immobilized affinity binders with binding affinity for different analyte molecules; and detecting binding of the analyte molecule-supramolecular structure complexes to binding sites of the array.

As provided herein, a method for detecting an analyte molecule present in a sample can include providing an array of supramolecular structures, wherein each individual supramolecular structure is immobilized on a respective binding site of a substrate. An individual supramolecular structure of the array includes a core structure comprising a plurality of core molecules; and a plurality of nucleic acid capture molecules linked to the core structure, wherein the nucleic acid capture molecules each comprise a same identification sequence distinguishable from identification sequences other supramolecular structures of the array. The method includes contacting the array with a pool of affinity binders linked to single-stranded nucleic acid tags with binding specificity for different identification sequences to cause the different affinity binders to bind to different binding sites of the array such that each binding site comprises a different subset of the pool of affinity binders. The method also includes contacting the sample with the array such that the analyte molecule binds to an individual affinity binder of the pool of affinity binders, wherein the analyte molecule is complexed with a supramolecular structure detection assembly and detecting binding of the analyte molecule to the individual binding site.

As provided herein, a method for detecting an analyte molecule present in a sample can include providing an array comprising single-stranded nucleic acids immobilized on the array such that an individual binding site of the array comprises a plurality of single-stranded nucleic acids comprising a same sequence that is distinguishable from sequences of other single-stranded nucleic acids immobilized on different binding sites of the array. The method includes contacting the array with a pool of affinity binders to cause the immobilized single-stranded oligonucleotides to capture a subset of the affinity binders at binding sites of the array and such that the captured affinity binders form capture molecules immobilized at respective binding sites and contacting the sample with the array such that the analyte molecule binds to an individual capture molecule, wherein the analyte molecule is complexed with a supramolecular structure detection assembly. The method also includes detecting binding of the analyte molecule to the individual binding site.

As provided herein, a method for detecting an analyte molecule present in a sample can include forming nanoballs or functionalized nanostructures. The nanoballs or functionalized nanostructures include a plurality of individual oligonucleotides, each oligonucleotide of the plurality being linked to a chemical moiety and each oligonucleotide being bound to complementary nucleic acids of the nanoball or functionalized nanostructure. The method includes providing a patterned array comprising a plurality of active sites; incubating the nanoballs or functionalized nanostructures with the patterned array to covalently link active groups of an individual active site to chemical moieties of the plurality of individual oligonucleotides to couple the individual the nanoball or functionalized nanostructure to the individual active site; denaturing the plurality of individual oligonucleotides from the complementary nucleic acids of the nanoball or functionalized nanostructure; washing the nanoball or functionalized nanostructure from the array to leave the plurality of individual oligonucleotides immobilized on the individual binding site, wherein the plurality of individual oligonucleotides are single-stranded; and contacting the array with a pool of affinity binders to cause the immobilized plurality of individual oligonucleotides to capture a subset of the affinity binders at the individual binding site.

As provided herein, a method for detecting an analyte molecule present in a sample can include providing a sample comprising analyte molecules and contacting the sample with a pool of supramolecular structures in solution to form analyte molecule-supramolecular structure complexes. An individual analyte molecule-supramolecular structure complex includes a core structure comprising a plurality of core molecules; an affinity binder linked to the core structure; a sample-specific barcode an analyte molecule of the analyte molecules bound to the affinity binder, wherein different supramolecular structures of the pool of supramolecular structures comprise different affinity binders with different binding affinity for other analyte molecules of the analyte molecules. The method includes pooling the analyte molecule-supramolecular structure complexes with other analyte molecule-supramolecular structure complexes, the other analyte molecule-supramolecular structure complexes being associated with respective different sample-specific barcodes; contacting the pooled analyte molecule-supramolecular structure complexes with an array, wherein binding sites of the array comprise respective immobilized affinity binders with binding affinity for different analyte molecules; detecting binding of the analyte molecule-supramolecular structure complexes to binding sites of the array; and associating the detected binding with the sample-specific barcode.

As provided herein, a method for detecting an analyte molecule present in a sample is disclosed. The method includes providing a plurality of supramolecular structures. Each supramolecular structure comprises a core structure comprising a plurality of core molecules; an antibody having binding affinity for an antigen and coupled to a nucleic acid capture strand; and a complementary strand to the nucleic acid capture strand, wherein the complementary strand is linked to the core structure and forms a duplex structure with the nucleic acid capture strand to couple the antibody to the core structure, wherein the plurality of supramolecular structures comprise different antibodies relative to one another with respective different binding affinities. The method includes contacting the plurality of supramolecular structures with a sample comprising the antigen such that the antigen binds to the antibody to form a complex; contacting the complex with a bead carrying a capture antibody having binding specificity for the antigen to form a sandwich structure; displacing the nucleic acid capture strand from the complementary strand using a displacing strand to release the core structure into solution, wherein the released core structure is not coupled to the antibody; and detecting the core structure.

In some embodiments, any method disclosed herein may include detecting a presence of and/or quantifying the concentration of the analyte molecule in the sample. In some embodiments, any method disclosed herein further comprising identifying the detected analyte molecule. In some embodiments, any method disclosed herein further comprising detecting the analyte molecule based on the signal when the analyte molecule is present in the sample at a count of a single molecule or higher.

In some embodiments, for any method disclosed herein, each core structure is a nanostructure. In some embodiments, for any method disclosed herein, the plurality of core molecules for each core structure are arranged into a pre-defined shape and/or have a prescribed molecular weight. In some embodiments, the pre-defined shape is configured to limit or prevent cross-reactivity with another supramolecular structure. In some embodiments, for any method disclosed herein, the plurality of core molecules for each core structure comprises one or more nucleic acid strands, one or more branched nucleic acids, one or more peptides, one or more small molecules, or combinations thereof. In some embodiments, for any method disclosed herein, each core structure independently comprises a scaffolded deoxyribonucleic acid (DNA) origami, a scaffolded ribonucleic acid (RNA) origami, a scaffolded hybrid DNA:RNA origami, a single-stranded DNA tile structure, a multi-stranded DNA tile structure, a single-stranded RNA origami, a multi-stranded RNA tile structure, hierarchically composed DNA or RNA origami with multiple scaffolds, a peptide structure, or combinations thereof.

The affinity binder of a supramolecular structure can be linked via chemical bond to the core structure. In some embodiments, the affinity binder of the solution-based supramolecular structure and/or immobilized affinity binders of the binding sites independently comprise a protein, a peptide, an antibody, an aptamer (RNA and DNA), a fluorophore, a darpin, a catalyst, a polymerization initiator, a polymer like PEG, or combinations thereof.

In some embodiments, for any method disclosed herein, a detectable signal comprises a barcode indicative of a particular affinity binder, e.g., of a binding site and/or a supramolecular structure. In some embodiments, each barcode provides a DNA signal or initiator signal corresponding to the affinity binder and providing information indicative of its specificity for an analyte molecule bound to the respective detector molecule. In some embodiments, the barcodes are analyzed using genotyping, qPCR, sequencing, or combinations thereof. In some embodiments, a plurality of analyte molecules in the sample are detected simultaneously through multiplexing. In some embodiments, for any method disclosed herein, the affinity binders as provided herein are configured for binding to one or more specific types of analyte molecules.

In some embodiments, for any method comprising using a plurality of supramolecular structures disclosed herein, each core structure of the plurality of supramolecular structures are identical to each other. However, the coupled affinity binders can vary for the plurality. Thus, in an embodiment, a pool of (e.g., a plurality of) supramolecular structures is identical except for a coupled affinity binder and, in some embodiments, an identification moiety (e.g., an affinity binder identification barcode nucleic acid sequence) that identifies the coupled affinity binder. For multiplexed samples detected on a single substrate or detection platform, the supramolecular structures associated with a particular sample can carry a sample-associated identification moiety (e.g., a sample barcode nucleic acid sequence) that identifies the sample.

In some embodiments, each supramolecular structure comprises a prescribed shape, size, molecular weight, or combinations thereof, which may reduce or eliminate cross-reactions between a plurality of supramolecular structures. In some embodiments, each supramolecular structure comprises only one affinity binder or multiple affinity binders. Where a supramolecular structure includes multiple affinity binders on a single core structure, the multiple affinity binders may all have a same binding specificity, e.g., all specifically bind a same analyte. In some embodiments, each supramolecular structure comprises a prescribed stoichiometry of the capture and detector molecules so as to reduce or eliminate cross-reactions between the plurality of supramolecular structures.

In some embodiments, the substrate comprises a solid support, solid substrate, a polymer matrix, or one or more beads. The substrate can include a plurality of binding sites patterned on the substrate and separated by interstitial surfaces of the substrate, wherein each binding site comprises a well, and wherein at least one surface of the well comprises a first chemical group and the interstitial surfaces comprise a second chemical group. A bead is loaded into each of the binding sites, wherein the first chemical group selectively binds to the bead and the second chemical group does not interact with or bind to the bead. Each bead comprises a plurality of single-stranded oligonucleotides immobilized on each bead such that an individual binding site of the array comprises a plurality of single-stranded oligonucleotides comprising a same sequence that is distinguishable from sequences of other single-stranded oligonucleotides immobilized on different binding sites of the array and affinity binders captured at binding sites of the array such that the captured affinity binders form capture molecules immobilized at respective binding sites. During detection, analyte molecules are bound to individual capture molecules at the individual binding site, wherein each analyte molecule is complexed with a supramolecular structure detection assembly. Detection of the detection assembly permits detection of the analytes bound to the substrate.

In some embodiments, a plurality of supramolecular structures are disposed on a substrate, such as a shaped or planar substrate, wherein the substrate comprises a plurality of binding sites, wherein each individual binding site is coupled to one or more affinity binders configured to bind to the same analyte molecule, e.g., such that an individual binding site is specific for an individual analyte molecule and different binding sites of the substrate have specificity for different analyte molecules. The disclosed embodiments also include sample preparation reagents, substrates, and detection systems for performing the disclosed methods.

In some embodiments, for any method disclosed herein, the sample comprises a complex biological sample. In some embodiments, for any method disclosed herein, the analyte molecule or molecules of a sample comprise a protein, a peptide, a peptide fragment, a lipid, a DNA, a RNA, an organic molecule, an inorganic molecule, complexes thereof, or any combinations thereof. In some embodiments, for any method disclosed herein, the sample comprises a biological particle or a biomolecule. In some embodiments, for any method disclosed herein, the sample comprises an aqueous solution comprising a protein, a peptide, a fragment of a peptide, a lipid, DNA, RNA, an organic molecule, a viral particle, an exosome, an organelle, or any complexes thereof. In some embodiments, for any method disclosed herein, the sample comprises a tissue biopsy, blood, blood plasma, Urine, Saliva, Tear, Cerebrospinal fluid, extracellular fluid, cultures cells, culture media, discarded tissue, plant matter, a synthetic protein, prions, a bacterial and/or viral sample or fungal tissue, or combinations thereof. The sample may be an environmental sample, such as a wastewater or soil sample. The sample may also be a nonbiological sample. In an embodiment, the sample may be a sample from a chemical process step, a sample of food or nutritional components, or packaging components.

The sample may be processed to release the analytes from cells or to otherwise prepare the sample for analysis prior to contacting the sample with the supramolecular structures in solution as provided herein.

### BRIEF DESCRIPTION OF THE DRAWINGS

Specific embodiments of the disclosed devices, delivery systems, or methods will now be described with reference to the drawings. Nothing in this detailed description is intended to imply that any particular component, feature, or step is essential to the invention.

FIG. 1 shows an example analyte-supramolecular structure complex after solution-based capture according to embodiments of the disclosure.

FIG. 2 shows an example workflow including solution-based single-molecule capture sample preparation according to embodiments of the disclosure.

FIG. 3 shows an example of a substrate for analyte detection according to embodiments of the disclosure.

FIG. 4 shows an example of a binding site of a substrate with a capture molecule and a captured analyte-supramolecular structure complex according to embodiments of the disclosure.

FIG. 5 shows an example of a substrate for analyte detection according to embodiments of the disclosure.

FIG. 6 shows a concatenated barcode nucleic acid structure for use with a substrate for analyte detection according to embodiments of the disclosure.

FIG. 7 shows an example of a substrate for analyte detection including the concatenated barcode nucleic acid structure of FIG. 6 according to embodiments of the disclosure.

FIG. 8 shows a nanoparticle structure for use with a substrate for analyte detection according to embodiments of the disclosure.

FIG. 9 shows an example of a substrate for analyte detection including the nanoparticle structure of FIG. 5 according to embodiments of the disclosure.

FIG. 10 shows an example of a substrate for analyte detection according to embodiments of the disclosure.

FIG. 11 shows an example of a bead for analyte detection according to embodiments of the disclosure.

FIG. 12 shows an example workflow for generating a substrate to accommodate beads for analyte detection according to embodiments of the disclosure.

FIG. 13 shows an example workflow including solution-based single-molecule capture sample preparation for multiplexed detection according to embodiments of the disclosure.

FIG. 14A shows steps of an experimental workflow for antigen detection including an IgG affinity binder complexed to a box origami to form a supramolecular structure according to embodiments of the disclosure.

FIG. 14B shows additional steps of the experimental workflow of FIG. 14A.

FIG. 15 shows different experimental subgroups assessed using the experimental workflow of FIGS. 14A-B.

FIG. 16 shows solution-based optical detection results with different antigen combinations of the three antigens used in the experimental workflow of FIGS. 14A-B.

FIG. 17 shows solution-based optical detection results with titration of TSH used in the experimental workflow of FIGS. 14A-B.

FIG. 18 shows solution-based optical detection results for antigen titration both with or without the presence of another antigen.

FIG. 19 shows solution-based optical detection results for monomer dimer, and trimer antigens.

FIG. 20 shows solution-based optical detection results for mixtures of unlabeled and labeled antigens.

FIG. 21 shows solution-based optical detection results for mixed or complex samples.

FIG. 22 shows solution-based optical detection results for titrated antigen samples

FIG. 23 shows a block diagram of an example analyte detection system according to embodiments of the disclosure.

### DETAILED DESCRIPTION

Disclosed herein are structures and methods for detecting one or more analyte molecules present in a sample. In some embodiments, the one or more analyte molecules are detected based on solution-based capture by one or more supramolecular structures. In some embodiments, the one or more supramolecular structures include or are linked to an affinity binder that specifically binds to an analyte present in a sample and in solution for single-molecule capture of the analyte. Binding of the analyte forms analyte molecule-supramolecular structure complexes in solution, and these complexes can be detected by a detection system as provided herein.

In embodiments, the complexed analyte is captured by an immobilized affinity binder associated with a substrate or other detection platform of a detection system. Capture of the complexes in turn immobilizes the complexes on the substrate, and one or more characteristics of the immobilized substrates, or their associated binding sites, can be characterized to characterize analytes in the sample. In an embodiment, the supramolecular structures includes a detectable moiety, such as a unique identifier (e.g., a nucleic acid sequence, a peptide, a polysaccharide, an acrydite) and/or a molecule that includes, interacts with, or that can be used to dock other molecules that can be detected (e.g., optically, electrically, magnetically). In some embodiments, the detectable moiety generates a DNA signal or other initiator signal, such that detection and quantification of binding of an analyte molecule-supramolecular structure complex to a binding site can be detected by amplification of the unique identifier of the supramolecular structure and/or a unique identifier of the binding site. In some embodiments, the supramolecular structure is linked to an enzyme that converts a substrate to an optically detectable signal. In some embodiments, the supramolecular structure is coupled to a sensor on the substrate to generate an electrically or magnetically detectable signal. In an embodiment, the supramolecular structure is a nucleic acid origami that is linked to or immobilized on a substrate. In an embodiment, the supramolecular structure carries the affinity binder via a barcode bridge or linker that include the unique identifier for the affinity binder and that links the affinity binder to a scaffold of the supramolecular structure.

In some embodiments, the disclosed techniques provide single molecule capture of analyte molecules in a complex sample. Use of the supramolecular structure as the capture entity permits specific identification and, in embodiments, detection of associated affinity binders via interaction with binding sites of a substrate. Thus, as provided herein, detectable analyte binding to an individual affinity binder among a pool of many different affinity binders on the substrate to generate assay results in which binding characteristics of an analyte pool of multiple different analytes are characterized. This in turn permits a sample having an uncharacterized composition of analytes to be analyzed for the presence and/or concentration of particular analytes of interest. For example, a human sample can be characterized to determine a presence and/or concentration of antibodies with binding specificity to particular antigens in a panel of antigen affinity binders, such that the affinity binders represent a known infectious disease antigen panel. The assay results may show positive binding results associated with a particular antigen, which is indicative of the presence of antibodies in the subject providing the sample. In another embodiment, the identity of analytes in the sample may be at least partially known, but their binding affinity may not be characterized for a particular pool of affinity binders. For example, the affinity binders can be a set of candidate drugs, and the analytes can be molecules in human blood. Binding of a drug candidate to such a protein can be used to assess bioavailability or potential off-target binding. The assay results may show positive binding results associated with a particular drug candidate that can in turn be mapped to a particular analyte, which is based on identification of particular detector binding (e.g., identifying binding by barcode identification in a detector molecule assembly that includes an antibody specific for the analyte).

While conventional detection protocols for analytes such as proteins may include detectable fluorescent signals generated by enzymes linked to detection antibodies as an indicator of binding, the disclosed techniques may additionally or alternatively provide amplified nucleic acid (or other initiator) signals from a unique identifier of the detector molecule assembly and from which sequence information can be determined or from which an optically detectable signal is released that corresponds to amplification (e.g., qPCR using a primer/probe set specific for the unique identifier). Thus, unique identity information for the captured complexes at particular binding sites of an array permits specific identification of the particular affinity binder that has captured an analyte in certain embodiments. The modular and customizable structure of the components of the supramolecular structure permits providing bulk or common core structures that are generally the same and barcoding individual supramolecular structures with a unique identifier during linkage of a particular affinity binder. Thus, the identification sequence is tied to only one specific affinity binder. Other detection techniques may include optical, magnetic, and or electrical detection techniques

Disclosed embodiments relate to analyte detection in which the analytes are present in a sample, such as a biological sample. In some embodiments, the sample comprises an aqueous solution comprising protein, peptides, peptide fragments, lipids, DNA, RNA, organic molecules, inorganic molecules, complexes thereof, or any combinations thereof. In some embodiments, the analyte molecules in the sample comprise protein, peptides, peptide fragments, lipids, DNA, RNA, organic molecules, inorganic molecules, complexes thereof, or any combinations thereof. In some embodiments, the analyte molecules comprise comprises intact proteins, denatured proteins, partially or fully degraded proteins, peptide fragments, denatured nucleic acids, degraded nucleic acid fragments, complexes thereof, or combinations thereof. In some embodiments, the sample is obtained from tissue, cells, the environment of tissues and/or cells, or combinations thereof. In some embodiments, the sample comprises tissue biopsy, blood, blood plasma, urine, saliva, a tear, cerebrospinal fluid, extracellular fluid, cultures cells, culture media, discarded tissue, plant matter, synthetic proteins, bacterial, viral samples, fungal tissue, or combinations thereof. In some embodiments, the sample is isolated from a primary source such as cells, tissue, bodily fluids (e.g., blood), environmental samples, or combinations thereof, with or without purification. In some embodiments, the cells are lysed using a mechanical process or other cell lysis methods (e.g., lysis buffer). In some embodiments, the sample is filtered using a mechanical process (e.g., centrifugation), micron filtration, chromatography columns, other filtration methods, or combinations thereof. In some embodiments, the sample is treated with one or more enzymes to remove one or more nucleic acids or one or more proteins. In some embodiments, the sample comprises intact proteins, denatured proteins, partially or fully degraded proteins, peptide fragments, denatured nucleic acids or degraded nucleic acid fragments. In some embodiments, the sample is collected from one or more individual persons, one or more animals, one or more plants, or combinations thereof. In some embodiments, the sample is collected from an individual person, animal and/or plant having a disease or disorder that comprises an infectious disease, an immune disorder, a cancer, a genetic disease, a degenerative disease, a lifestyle disease, an injury, a rare disease, an age-related disease, or combinations thereof.

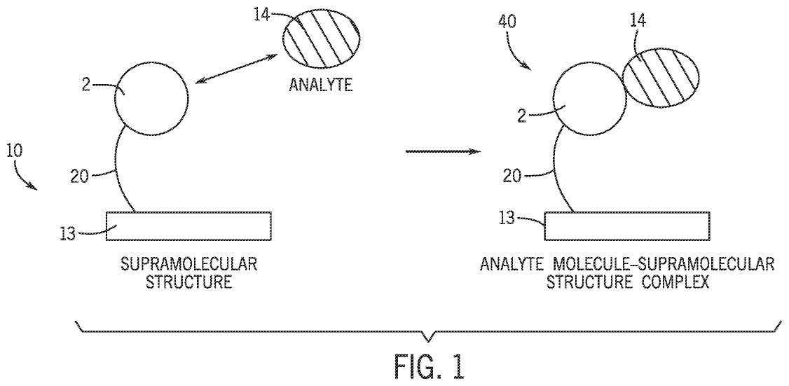

The disclosed techniques harness single-molecule binding of affinity molecules linked to supramolecular structures. The single-molecule binding of an analyte molecule forms a complex as shown in FIG. 1, which is detectable by a detection system according to the disclosed techniques. The complex formation occurs in solution. FIG. 1 provides an exemplary embodiment of a supramolecular structure 10 comprising a core structure 13 and an affinity binder 2. In some embodiments, the supramolecular structure 10 comprises one or more affinity binders 2. In an embodiment, the supramolecular structure 10 may refer to a complex that includes a core structure 13 and the affinity binder 2. In an embodiment, the supramolecular structure 10 may refer the core structure 13, and the supramolecular structure 10 may or may not include the affinity binder 2.

Accordingly, provided herein are supramolecular structures 10. In some embodiments, the supramolecular structure 10 is a programmable structure that can spatially organize molecules. In some embodiments, the supramolecular structure 10 comprises a plurality of molecules linked together. In some embodiments, the plurality of molecules of the supramolecular structure 10 interact with at least some of each other. In some embodiments, the supramolecular structure 10 comprises a specific shape, e.g., a substantially planar shape that has a longest dimension in an x-y plane. In some embodiments, the supramolecular structure 10 is a nanostructure. In some embodiments, the supramolecular structure 10 is a nanostructure that comprises a prescribed molecular weight based on the plurality of molecules of the supramolecular structure 10. In some embodiments the plurality of molecules are linked together through a bond, a chemical bond, a physical attachment, or combinations thereof. In some embodiments, the supramolecular structure 10 comprises a large molecular entity, of specific shape and molecular weight, formed from a well-defined number of smaller molecules interacting specifically with each other. In some embodiments, the structural, chemical, and physical properties of the supramolecular structure 10 are explicitly designed. In some embodiments, the supramolecular structure 10 comprises a plurality of subcomponents that are spaced apart according to a prescribed distance. In some embodiments, at least a portion of the supramolecular structure 10 is rigid. In some embodiments, at least a portion of the supramolecular structure 10 is semi-rigid. In some embodiments, at least a portion of the supramolecular structure is flexible. In an embodiment, the supramolecular structure 10 is at least 50-200 nm in one dimension. In an embodiment, the supramolecular structure 10 is at least 20 nm long in any dimension.

In some embodiments, the core structure 13 is a polynucleotide structure, a protein structure, a polymer structure, or a combination thereof. In some embodiments, the core structure 13 comprises one or more core molecules linked together. In some embodiments, the one or more core molecules comprise 2, 3, 4, 5, 6, 7, 8, 9, 10, 20, 50, 100, 200 or 500 unique molecules that are linked together. In some embodiments, the one or more core molecules comprises from about 2 unique molecules to about 1000 unique molecules. In some embodiments, the one or more core molecules interact with each other and define the specific shape of the supramolecular structure. In some embodiments, the plurality of core molecules interact with each other through reversible non-covalent interactions.

In some embodiments, the specific shape of the core structure 13 is a three-dimensional (3D) configuration. In some embodiments, the one or more core molecules provide a specific molecular weight. For example, all core structures 13 of supramolecular structures 10 of a plurality may have a same configuration, size, and/or weight, but may different in their attached linker sequences and attached affinity binders 2. However, excluding different linkers 20 and affinity binders 2, the supramolecular structures 10 of a plurality may be otherwise identical. In some embodiments, the core structure 13 is a nanostructure. In some cases, the one or more core molecules comprise one or more nucleic acid strands (e.g., DNA, RNA, unnatural nucleic acids), one or more branched nucleic acids, one or more peptides, one or more small molecules, or combinations thereof. In some embodiments, the core structure 13 comprises an entirely polynucleotide structure. In some embodiments, at least a portion of the core structure 13 is rigid. In some embodiments, at least a portion of the core structure 13 is semi-rigid. In some embodiments, at least a portion of the core structure 13 is flexible. In some embodiments, the core structure 13 comprises a scaffolded deoxyribonucleic acid (DNA) origami, a scaffolded ribonucleic acid (RNA) origami, a scaffolded hybrid DNA/RNA origami, a single-stranded DNA tile structure, a multi-stranded DNA tile structure, a single-stranded DNA origami, a single-stranded RNA origami, a single-stranded RNA tile structure, a multi-stranded RNA tile structures, a hierarchically composed DNA and/or RNA origami with multiple scaffolds, a peptide structure, or combinations thereof. In some embodiments, the DNA origami is scaffolded. In some embodiments, the RNA origami is scaffolded. In some embodiments, the hybrid DNA/RNA origami is scaffolded. In some embodiments, the core structure 13 comprising a DNA origami, RNA origami, or hybrid DNA/RNA origami that comprises a prescribed two-dimensional (2D) or 3D shape.

In an embodiment, the core structure 13 is a nucleic acid origami that has at least one lateral dimension between about 50 nm to about 1μ. In an embodiment, the nucleic acid origami has at least one lateral dimension between about 50 nm to about 200 nm, about 50 nm to about 400 nm, about 50 nm to about 600 nm, about 50 nm to about 800 nm, about 100 nm to about 200 nm, about 100 nm to about 300 nm, about 100 nm to about 400 nm, about 100 nm to about 500 nm, about 200 nm to about 400 nm by way of example. In an embodiment, the nucleic acid origami has at least a first lateral dimension between about 50 nm to about 1μ. and a second lateral dimension, orthogonal to the first, between about 50 nm to about 1μ. In an embodiment, the nucleic acid origami has a planar footprint having an area of about 200 nm² to about 1μ².

As shown in FIG. 1, in some embodiments, the core structure 13 is configured to be linked to an affinity binder 2. In some embodiments, the affinity binder 2 is immobilized with respect to the core nanostructure 13 when linked thereto. However, the core structure 13 may be in solution and thus, not immobilized with respect to a sample or reaction vessel. As shown in FIG. 1, in some embodiments, the affinity binder 2 is linked to the core structure 13 through the linker 20. In some embodiments, the linker 20 comprises a polymer that comprises a nucleic acid (double or single-stranded DNA or RNA) of a specific sequence that is associated with the linked affinity binder 2. Thus, the sequence of the nucleotide linker 20 uniquely identifies the affinity binder 2 among a pool of different affinity binder 2. The barcode may be at least 6 nucleotides, and may be 6-50 nucleotides. In FIG. 1, the supramolecular structure 10 binds to an analyte molecule 14 having binding specificity for the affinity binder 2 to form an analyte molecule-supramolecular structure complex 40.

In some embodiments, any number of the one or more core molecules 13 comprises one or more linkers 20 configured to form a linkage with the affinity binder 2. In some embodiments, the linker 20 is linked to one or more core molecules of the core structure 13 through a chemical bond. In some embodiments, the linker 20 may include a core reactive molecule. In some embodiments, each core reactive molecule independently comprises an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, at least one of the one or more core linkers comprises a DNA sequence domain.

In some embodiments, the core structure 13 is linked to the affinity binder 2 at a prescribed location on the core structure 13.

In some embodiments, the affinity binder 2 comprises a protein, a peptide, an antibody, antibody-derived reagents, an aptamer (RNA and DNA), a fluorophore, a nanobody, a darpin, a catalyst, a polymerization initiator, a polymer like PEG, an organic molecule, small molecule, a pharmaceutical compound, a candidate pharmaceutical compound, a synthetic molecule, or combinations thereof. In some embodiments, a single affinity binder 2 is linked to the core structure 13. In some embodiments, a plurality of affinity binders 2 are linked to a core structure 13. For example, different affinity binders 2 on a same core structure 13 may represent different binding sites for a same analyte molecule or may bind different analyte molecules of a multi-molecule complex, e.g., of a protein-protein complex. In another example, multiple same affinity binders 2 may be present on a core structure 13.

In some embodiments, each component of the supramolecular structure 10 may be independently modified or tuned. In some embodiments, modifying one or more of the components of the supramolecular structure 10 may modify the 2D and 3D geometry of the supramolecular structure itself. In some embodiments, modifying one or more of the components of the supramolecular structure may modify the 2D and 3D geometry of the core structure 13. In some embodiments, such capability for independently modifying the components of the supramolecular nanostructure enables precise control over the organization of one or more supramolecular structures.

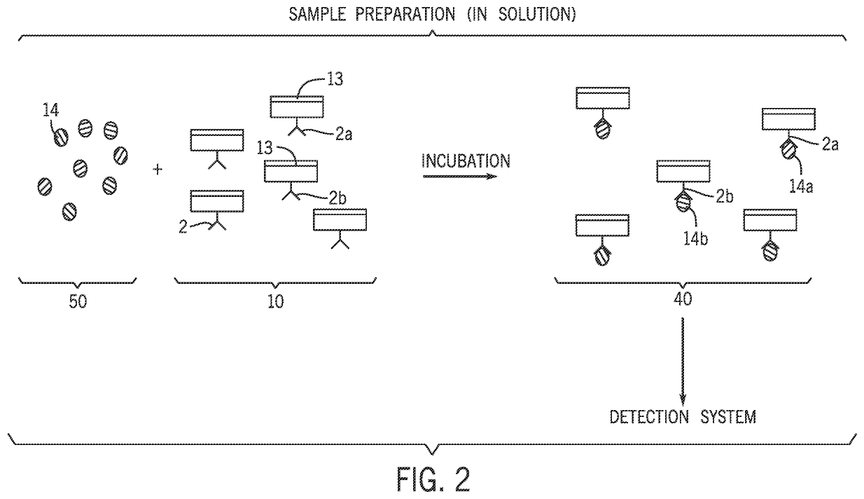

As described herein, in some embodiments, one or more supramolecular structure complexes 40 enable the detection of one or more analyte molecules in a sample. FIG. 2 is a schematic illustration of a workflow for forming the analyte molecule-supramolecular structure complexes 40 in solution. A pool of supramolecular structures 10, with associated affinity binders 2 that represent a panel or set of affinities for respective different analytes, is contacted with a sample 50 that includes a plurality of different analyte molecule 14. The analytes 14 in the sample 50 can be uncharacterized or unknown analytes 14. In embodiments, the sample 50 may include one or more control analytes 14.

When the individual analyte molecule 14 and individual affinity binder 2 have binding specificity for one another, the analyte molecule 14 associates with the affinity binder 2 to form an individual analyte molecule-supramolecular structure complex 40. The reaction conditions permit binding of the analyte molecules 14 to specific affinity binders 2. As provided herein, binding specificity may refer to an interaction between the analyte molecule 14 and the affinity binder 2 that remains intact under the reaction conditions and after washing or removal steps for unbound reagents. Binding specificity may include formation of a covalent or non-covalent bonds, ionic bonds, dipole interactions, hydrophilic or hydrophobic interactions, complementary nucleic acid binding, etc. Specific binding may refer to binding to an analyte molecule 14 that binds only to a particular affinity binder 2 and not to other affinity binders 2. Thus, certain affinity binders 2 of the pool of supramolecular structures 10 bind to certain analyte molecules 14 (e.g., binding between a first analyte molecule 14a and a first affinity binder 2a or binding between a second analyte molecule 14b and a first affinity binder 2b). Certain affinity binders 2 may have no available binding partners in a given sample 50 and, therefore, do not bind to any analyte molecule 14 with specificity.

Any unbound analytes 14 can be removed after formation of the complexes 40 before the complexes are provided to a detection system, as provided herein. However, in other embodiments, no wash step is performed. Unbound analytes 14 in solution may be permitted to interact with the detection system, but are unlikely to specifically bind at binding sites and will not generate a detectable signal because they carry no supramolecular structure 10.

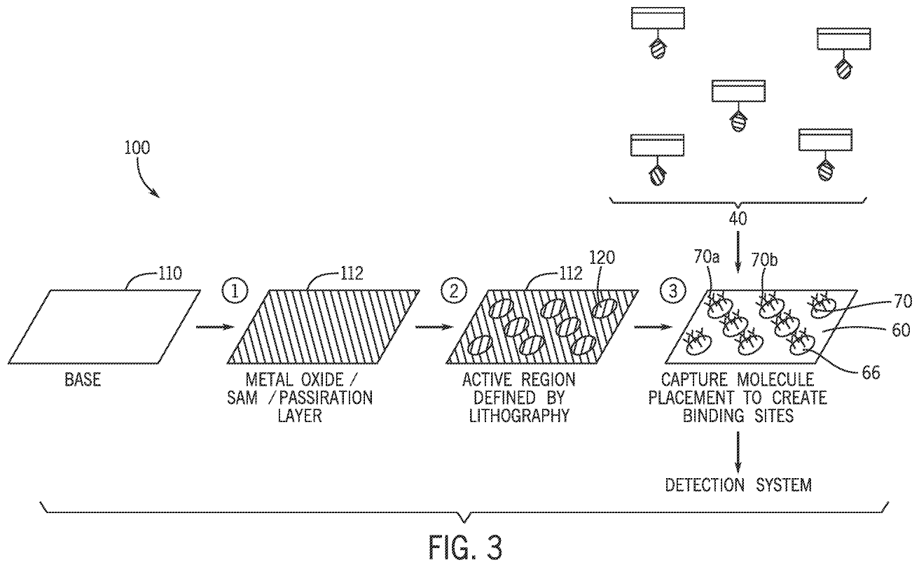

Analytes 14 of the disclosed complexes 40 may be detected based on interaction with a patterned substrate 60 having an array of binding sites 66 distributed on or in the substrate 60, as generally illustrated in FIGS. 3-13. A substrate 60 may include a defined set of micropatterned binding sites 66 functionalized by having immobilized capture molecules 70.

In some embodiments, the binding sites 66 are micropatterned on the planar substrate 60. In some embodiments, the binding sites 66 on the surface are in a periodic pattern. In some embodiments, the binding sites 66 on the surface are in a non-periodic pattern (e.g., random). In some embodiments, a minimum distance is specified between any two binding sites 66. In some embodiments, the minimum distance between any two binding sites 66 is at least about 200 nm. In some embodiments, the minimum distance between any two binding sites 66 is from at least about 40 nm to about 5000 nm. In some embodiments, the geometric shape of the binding sites 66 comprises a circle, square, triangle or other polygon shapes. In some embodiments, an individual binding site 66 is 20-200 nm in diameter. The substrate 60 may be patterned as generally discussed U.S. Provisional Application No. 63/119,316, filed on Nov. 30, 2020, which is incorporated herein by reference.

The substrate 60 may include a glass or silicon wafer having one or more silicon dioxide, silicon nitride, graphene or silicon carbide layers. In an embodiment, each binding site 66 accommodates multiple capture molecules 70 of a same type at each binding site 66, with different binding sites 66 having different capture molecule specificity or differential chemistry. The patterned substrate 60 may be fabricated through lithography processes. Further, embodiments of the disclosed techniques may include one or more regeneration steps that remove a bound or “used” complex 40 from the substrate 60 to permit binding of new complexes 40 in a subsequent reaction.

In some embodiments, the substrate 60 may include fiduciary markers (not shown) having geometric features defined on a surface to be used as reference features for other features on the substrate 60. In some embodiments, the planar substrate 60 comprises structures that facilitate detection, such as optical or electrical devices like FET, ring resonators, photonic crystals or microelectrode, to be defined prior to the formation of the binding sites 66.

FIG. 3 provides an exemplary illustration of forming a substrate 60 used for detecting analyte molecules in a sample using a surface based assay that uses capture molecules 70 (illustrated as antibodies, but that may be any suitable capture molecule to pull down complexes 40, as described herein. In an embodiment, the capture molecule 70 is an affinity binder as generally disclosed herein. In FIG. 3, a patterned substrate 60 with multiple binding sites 66 functionalized with or linked to individual capture molecules 70 can be formed according to the method 100. A substrate base layer 110 is provided. A passivation layer 112 is grown, assembled, or deposited on the base layer 110 that can be selectively passivated. The passivation layer or layers 112 may include silicon nitride, graphene, quartz, metal, gold, silver, platinum, palladium, PDMS, polymer film, or combinations thereof. The passivation layer 112 may be graphene, aluminum oxide, HfO₂, Cr₂O₃ (Chromium oxide), Titanium oxide, Tantalum oxide, metal oxides, silicon dioxide (SiO₂) or combinations thereof. The passivation layer 112 may be a self-assembled polymer, such as a polyacrylamide.

The passivation layer 112 is patterned, e.g., by removing portions of the top layer 112, to expose locations 120 of the base layer 110 that will correspond to binding sites 66 of the substrate 60. The patterning may be photolithography, e-beam lithography, nanoimprinting, polymer spin coating, optical patterning, plasma activation, acid/base treatment, or other patterning modalities. The exposed locations 120 may be activated by chemical or plasma treatment to yield different reactive groups, depending on the individual chemistry of these layers.

Capture molecules 70 can be coupled to the activated locations 120 to generate the binding sites 66. In the illustrated example, the capture molecules 70 can be printed on the binding site 66. Each individual binding site 66 is printed with a pre-selected capture molecule 70. However, other attachment or coupling techniques to link the capture molecules to the binding sites 66 are also contemplated. In an embodiment 1) each binding site includes multiple (e.g., two or more) capture molecules 70 and 2) all of the capture molecules 70 at an individual binding site have a same binding specificity for a particular analyte 14.

The capture molecules 70 may include one or more affinity binders, supramolecular structures, nucleic acids, as provided herein. Each binding site 66 of the substrate 60 includes a plurality of capture molecules 70 having a same binding specificity per individual binding site 66. Further, neighboring or different binding sites 66 can have different binding specificity such that first captures molecules 70a have different binding specificity than second capture molecules 70b.

The capture molecules 70 immobilized at each binding site 66 (and not present at non-binding site location on the substrate 60) may comprise a protein, a peptide, an antibody, an aptamers (RNA and DNA), a fluorophore, a nanobody, a darpin, a catalyst, a polymerization initiator, a polymer like PEG, an organic molecule, or combinations thereof.

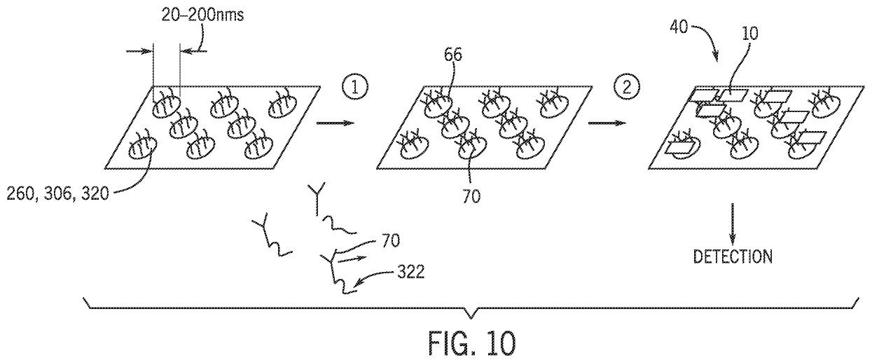

Each binding site 66 may be coupled to a barcode or unique identification sequence that can be read out either via sequencing, amplification, or via hybridization assay as part of detection. A map of capture molecules 70 immobilized on the substrate 60 and with the spatial locations on the substrate 60 can be obtained prior to performing the assay and performed as quality control of substrate 60. The map can be stored in an analyte detection system (see FIG. 10) as provided herein and used to generate a report of positive binding events to provide analyte information for a sample.

The substrate 60, once formed, can be used in an analyte capture step after sample preparation (as shown in FIG. 2) to provide complexes 40. In some embodiments, the complexes 40 are contacted with the planar substrate using a flow-cell. In some embodiments, the complexes 40 are incubated on the substrate 60 having capture molecules 70 attached to the binding sites 66. In some embodiments, the incubation period may be from about 30 seconds to about 24 hours. In some embodiments, the incubation period may be from about 30 seconds to about 1 minute, from about 1 minute to about 5 minutes, from about 5 minutes to about 30 minutes, from about 30 minutes to about 1 hr, from about 1 hr to about 5 hours, from about 5 hours to about 12 hours, from about 12 hours to about 24 hours, from about 24 hours to about 48 hours.

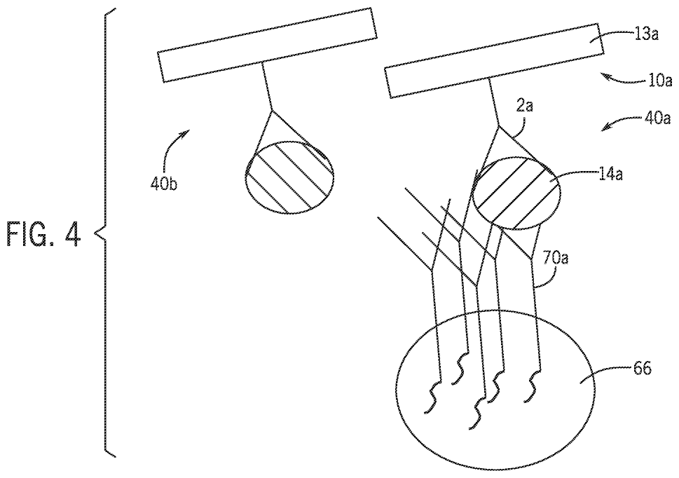

In some embodiments, the analyte molecules 14 of the complexes 40 interact with the capture molecules 70 on the binding sites 66. FIG. 4 shows an example binding site 66 with captures molecules 70a that have specificity for a particular analyte 14a of a complex 40a. After the interaction, binding can be detected via signal generation. Unbound complexes 40b may be washed off prior to detection. The capture molecules 70a and the respective analyte molecules 14a may also have binding specificity to one another. Because the analyte molecules 14a form complexes 40a in solution before contact with the substrate 60, the capture molecule 70a, analyte 14a, and affinity binder 2a (linked to a core structure 13a) can form a sandwich-like binding arrangement as shown in FIG. 4. Thus, the capture molecule 70a and the affinity binder 2a can both have specificity for the same analyte 14a. However, the capture molecule 70a and the affinity binder 2a may bind at different sites on the analyte 14a.

After these workflow steps, various bound complexes 40 remain on the array, each bound to respective capture molecules 70 and may be subjected to various detection protocols to associate the analyte 14 to a particular supramolecular structure identity, which in turn is associated with a known affinity binder 2 and at a particular binding site 66, that may in turn by associated with a unique identifier, e.g., a barcode sequence. Thus, detection permits characterization of analyte-affinity binder binding.

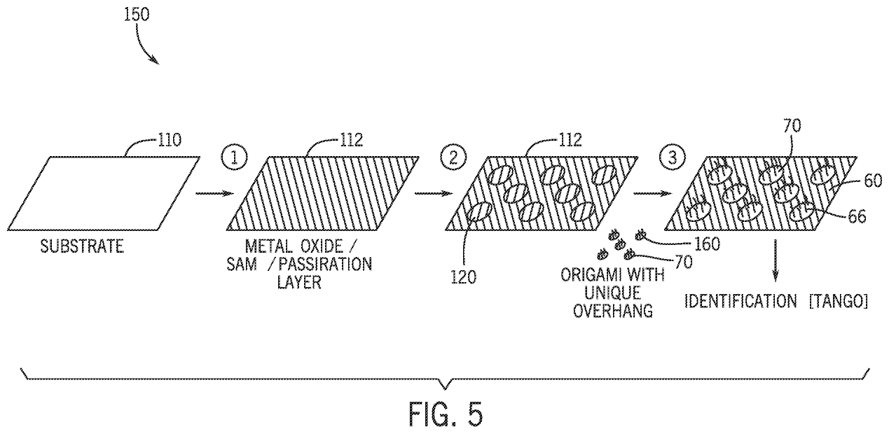

In some embodiments, as shown in a workflow 150 FIG. 5, the binding sites 66 are functionalized via a single supramolecular structure 160 that is placed at an activated location 120 to form a binding site 66. The binding sites 66 may be activated as generally disclosed with respect to FIG. 3 prior to placement of the supramolecular structures 160. The supramolecular structure 160 may be generally arranged as the supramolecular structure 10 as provided herein and may include a core structure 13 comprising a DNA origami, wherein the supramolecular structures 10 is attached onto each of the binding sites using DNA origami placement technique that may include linkage via an anchor molecule. In some embodiments, DNA origami placement comprises a directed self-assembly technique for organizing individual DNA origami (e.g., a core structure) on the binding site 66. In some embodiments, the planar substrate 60 could be stored for a significant period after this step, in a clean environment.

In the depicted embodiment, the supramolecular structure 160 is provided assembled and with capture molecules 70 already linked. Each individual supramolecular structure with respective different analyte specificity can be formed separately, e.g., in separate reaction tubes. Multiple capture molecules 70 are linked to a core structure 13 of the supramolecular structure 160. The capture molecules 70 of a single supramolecular structure 160 at a single binding site 66 all have a same binding specificity. Further, each supramolecular structure 160 includes a barcode or other identification sequence that uniquely identifies a type of associated capture molecule 70.

Placement of the preformed supramolecular structure 160 can be directional, such that the capture molecules 70 are positioned “right side up” on the binding site 66. In other embodiments, the supramolecular structures 160 are all generally the same, and are placed at random on individual binding sites. After placement at different binding sites, the unique identifying sequences and associated capture molecules 70 are linked.

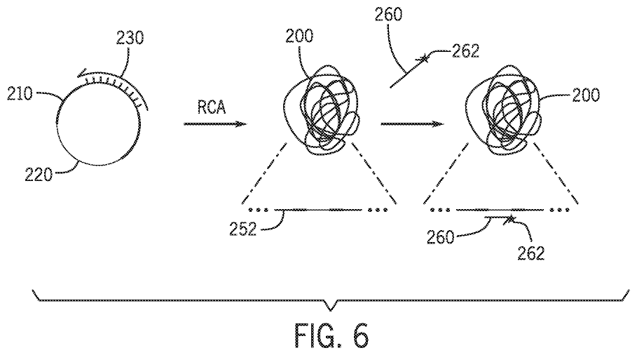

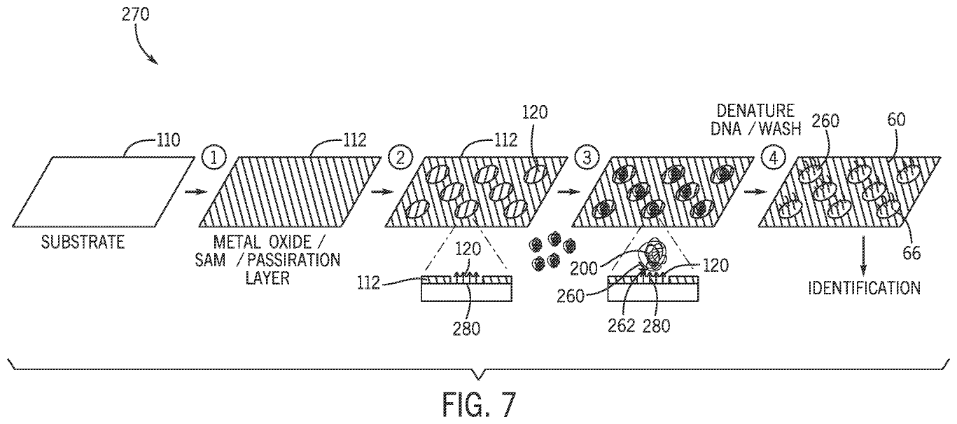

FIGS. 6-7 show steps in formation of a planar substrate 60 in which the binding sites 66 are functionalized via binders of a concatenated nucleic acid product 200. FIG. 5 shows steps in forming the concatenated nucleic acid product 200 from a circular template 210. The circular template includes a barcode sequence 220, which uniquely distinguishes the circular template 210 from other circular templates 210. Rolling circle amplification with a strand-displacing polymerase that extends a primer 230 generates the single-stranded rolling circle amplification product that includes concatenated nucleic acids with repeating units 252. Single-stranded probes 260 with active groups 262 hybridize to a complementary to the barcode sequence 220, and bind at multiple places on the concatenated nucleic acid product 200 where the barcode sequence 202 repeats as part of the repeating unit 252. Accordingly, the end product of the rolling circle amplification for binding site generation is the concatenated nucleic acid product 200 with multiple bound probes 260 and associated active groups 262. In an embodiment, formation of the substrate as provided herein includes creating a plurality of products 200 from respective different templates 210, whereby each product 200 has a distinguishable sequence based on the different templates 210. The respective different products 200 can be formed in different reaction vessels. However, in an embodiment, the different templates 210 can be pooled to generate pooled products 200. Because, as provided in FIG. 7, each product 200 associates generally in a 1:1 ratio with a binding site 66, a pooled population of the products 200 may nonetheless be distributed about the substrate 60. However, generating the products 200 independently may prevent differences in amplification biases that result in unequal production of products 200 from certain templates 210, which may in turn result in overrepresentation of certain template sequences on the binding sites 66.

Each template 210 includes a sequence that can be associated with or keyed to a particular capture molecule 70 as a barcode or unique identifier of the capture molecule 70 as provided herein.

As shown in a workflow 270 of FIG. 7, active sites 120 can be created as generally disclosed with respect to FIGS. 3-5. The binding sites 66 are functionalized via placement of a single concatenated nucleic acid product 200 with multiple bound probes 260 at activated locations 120 to form a binding site 66. The active sites 120, as shown, include active molecules 280 surrounded by the passivation layer 112. The single-stranded concatenated nucleic acid product 200 is a nanoball that is generally sized so that only one product 200 is accommodated on a single active site 120. The association of the product 200 with the active site during formation of the binding sites 66 is via the active groups 262 of the bounds (hybridized) probes 260. The interactions of the probes 262 with the active molecules can be via NHS-ester, thiol, DBCO, azide, maleimide interactions. For example, in one embodiment, the maleimide group reacts specifically with sulfhydryl groups when the pH of the reaction mixture is between 6.5 and 7.5; the result is formation of a stable thioether linkage. Accordingly, in an embodiment the active group 262 can be a maleimide reagent, and the active molecule 280 can be a sulfhydryl. The active group 262 and the active molecule 280 are reacted to form a stable conjugate thioether bond. Thus, the probes 260 are immobilized on the active sites 120. The nanoball product can be removed by denaturing from the probes 260 at denaturing temperatures and washing. This step leaves the probes 260 immobilized at the binding sites 66. The probes 260 all have a same sequence along at least a portion of the oligonucleotides. In an embodiment, the probes 260 all have a same sequence relative to one another within the binding site 66 but have a different sequence relative to probes 260 bound at some, most, or all other binding sites. In an embodiment, probes 260 of an individual binding site have at least 85% identity, at least 90% identity, at least 95% identity, or at least 99% identity with one another. Probes 260 between different binding sites 66 may have less than 50% sequence identity. Because the nanoball products 200 are generated from circular template 210 (see FIG. 6), different templates 210 having different sequences generate correspondingly different nanoball products 200. Thus, probes 260 that are complementary to the repeating units 252 of the different products 200 have different sequences based on the original template sequences.

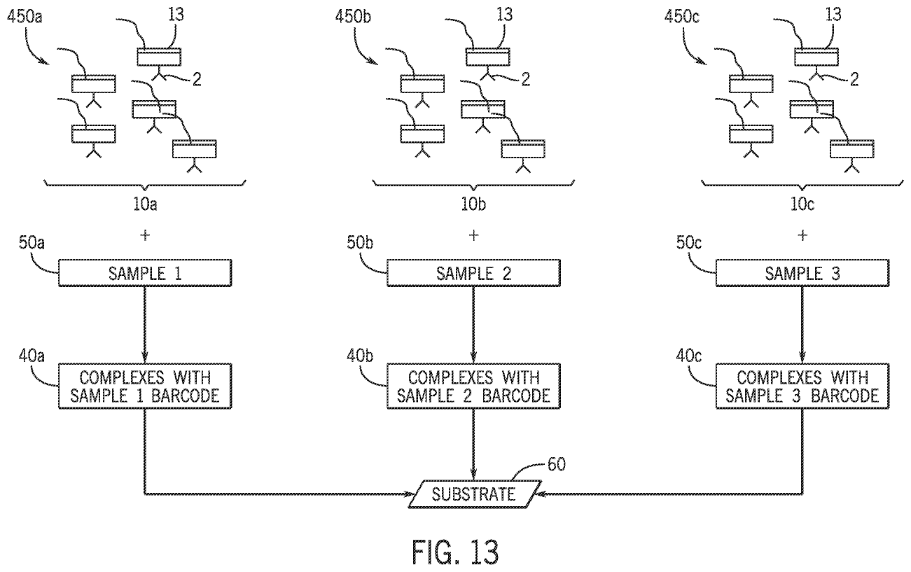

The probes 260, once immobilized, can be sequenced, amplified, or detected via tagged complementary oligonucleotides to map the binding sites 66 and their respective immobilized probes 260. The map can be stored in the detection system (FIG. 13).

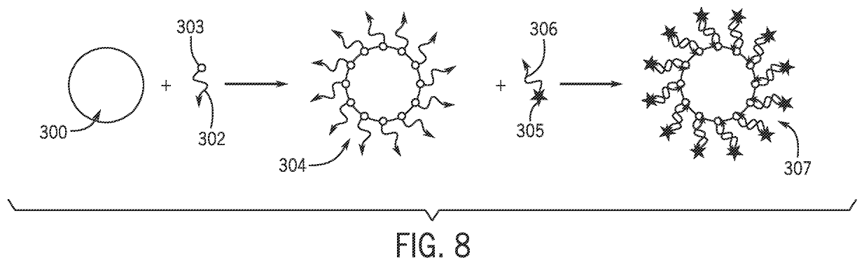

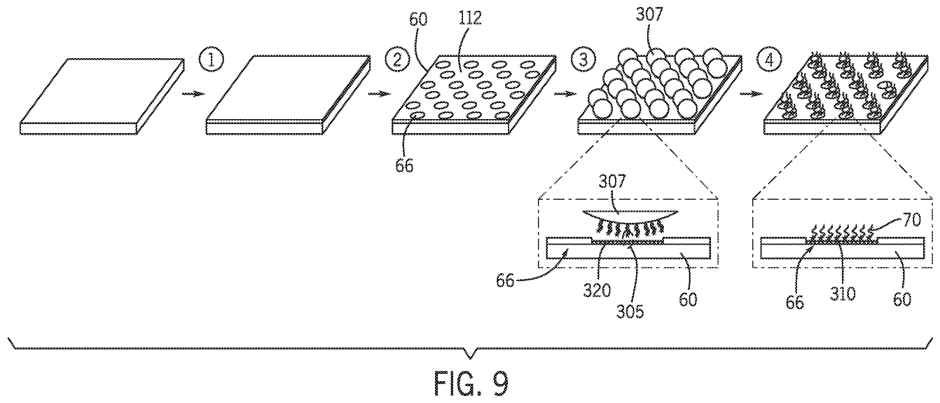

FIGS. 8-9 shows an alternate structural arrangement for capture molecule placement on binding sites 66 of the substrate 60. In FIG. 8, a nanoparticle 300, which may be made from a polymer hydrogel, cross-linked polymer or inorganic material is incubated with a single stranded oligonucleotide 302 having a chemical moiety 303 that can covalently bond to the nanoparticle 300. Subsequently, the nanoparticle 300 that has been functionalized with the single-stranded oligonucleotide 302 is incubated with a second single-stranded oligonucleotide 306 that has a second chemical moiety 305 to result in a functionalized nanoparticle 307 with immobilized double stranded DNA and carrying a chemical moiety 305. Accordingly, oligonucleotides 302, 306 interact on the basis of complementarity to form the double-stranded DNA, with the chemical moiety being coupled to the nanoparticle 307 via hybridization. Examples of the chemical moieties 303, 305 include, but are not limited to, thiol, amine, DBCO, maleiamide, azide, NHS-ester.

The functionalized nanoparticle 307 in FIG. 8 can be used to create monoclonal clusters of ssDNA on patterned substrates 60 by placement of the nanoparticles 307, covalent linkage to a surface, and subsequent removal/denaturation. Steps 1 and 2 to pattern the substrate 60 may be performed as generally disclosed herein (e.g., see FIG. 3). In an embodiment, the workflow involves first passivating the surface and then lithographically patterning following protocols as provided herein to to result in a surface with two chemical characteristics, one within the binding sites 66 and the other forming the passivating background 112. In step 3, the functionalized nanoparticle 307 of FIG. 8 is incubated on the patterned substrate 60 to result in organization of the nanoparticles 307 within the binding sites 66. The driving force of this assembly is the interaction between the nanoparticle 307 and the binding site 66. The nanoparticle 307 is sized so that there is generally a single nanoparticle 307 organized or placed at a single binding site 66. The chemical moiety 305 coupled to the nanoparticle 307 is covalently linked to the surface 310 of the binding site 66. Finally, the nanoparticles 307 as well as the complementary nucleic acid 302 nucleic acid on the nanoparticle is denatured to separate the oligonucleotide strands 302, 306 to result in a patterned surface in which each binding site has multiple copies of an identical ssDNA 306. The single-stranded DNA (or RNA) 306 is covalently linked to the surface 310 via the chemical moiety 305.

FIG. 10 shows coupling of capture molecules 70, shown as antibodies, to single-stranded oligonucleotides 320 placed at individual binding sites 66. The single-stranded oligonucleotides 300 may be probes 260 coupled via active groups 262 as shown in FIG. 7. The single-stranded oligonucleotides 300 may be oligonucleotides 306 coupled via moieties 305 as shown in FIG. 9. The single-stranded oligonucleotides 320, which are immobilized on the binding sites 66, may directly serve as capture molecules 70 (e.g. via hybridization). However, in embodiments, the unique sequences of the oligonucleotides 320 may be used to design complementary tags that are coupled to capture molecules 70 via hybridization in a binding site-specific manner. The single-stranded oligonucleotides 320 may, in embodiments, be directly printed on the binding sites 66, as shown in FIG. 3. The single-stranded oligonucleotides 320 at an individual binding site 66 all have a same sequence or have a sequence having at least 85% identity, at least 90% identity, at least 95% identity, or at least 99% identity. The capture molecules 70 are coupled to the oligonucleotides 300 by tags 290 that include complementary sequences. Each individual capture molecule 70, or type of capture molecule 70 having binding specificity for an individual analyte, is coupled to a tag 322 having a unique sequence distinguishable from other sequences of other tags 322 coupled to different capture molecules 70 having different specificity. The tags 322 are designed based on the known sequences of the oligonucleotides 320. Thus, a pool of different capture molecules 70 with respective unique tags 322 can be contacted with the binding sites 66 including the single-stranded oligonucleotides 320.

As shown, each binding site 66 can be coupled to multiple capture molecules 70, which in turn can interact with one, two, three or more complexes 40 via analyte binding to the capture molecules 70. The presence of the supramolecular structure 10 on the complexes keeps the average molecules per binding site low. However, the presence of multiple capture molecules 70 of a same specificity at each binding site 66 promotes strong specific binding of the analytes.

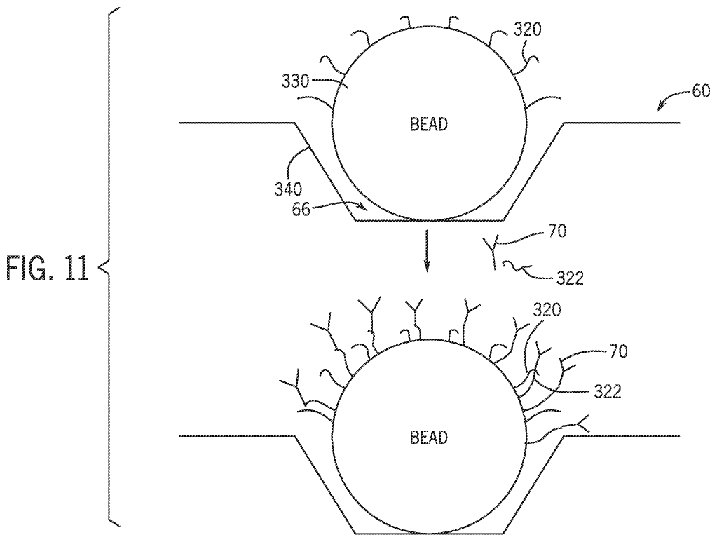

FIG. 11 is an embodiment in which the capture molecules 70 are coupled to a bead 330. The bead 330, in the illustrated embodiment, is disposed within a well 340 of the substrate 60 that is sized and shaped so that an individual binding site 66 formed by the well 340 accommodates a single bead 330. However, it should be understood that the beads 330 may be in solution and not associated with a substrate. Each bead is functionalized with unique oligonucleotides 320 all having a same or generally a same sequence as provided herein, and capture molecules 70 having a tag 322 hybridize to the oligonucleotides 320 only when they are complementary sequences.

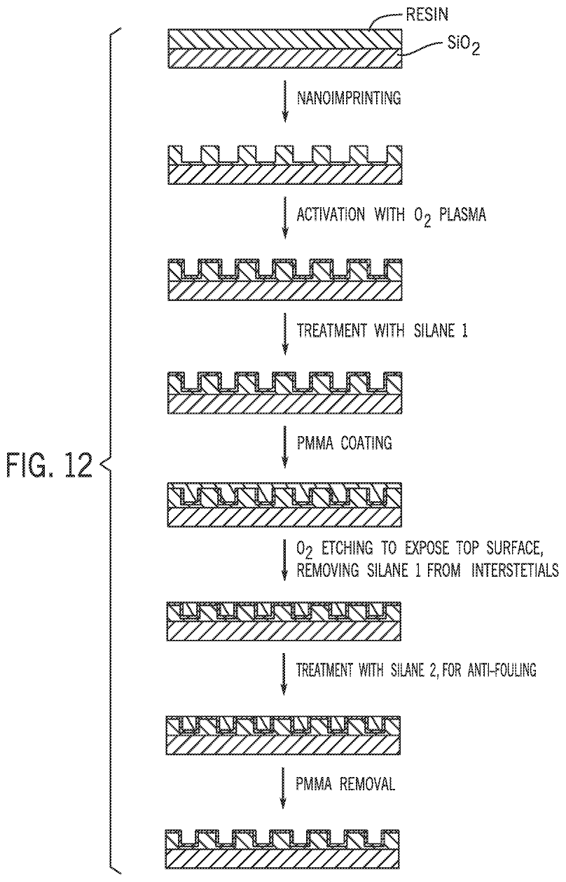

FIG. 12 shows an example of binding site formation (e.g., for binding sites 66 as in FIGS. 10-11). The binding sites 66 may be used to load beads 330 carrying a plurality of analytes 2 (e.g., DNA, RNA, or proteins/peptide as discussed herein) into lithographically defined nano/micro wells. The illustrated work flow creates an end product 350 (e.g., the substrate 60) with nano/micro wells 360 having an exterior surface that includes a first chemical or functional group on the inner walls and bottom of the wells 390 and another, different, chemical group having different reactivity on interstitial surfaces 362. Further, the first chemical group is designed such that it would interact (electrostatically or covalently) with the beads 330 while the other chemical group would not interact with the bead 300. In such a situation when the beads 330 (are incubated with the substrate 350 they would selectively go bind inside the well 360, while none would bind to the interstitials 360. The size or diameter of beads 330 will may be, in an embodiment, from 20 nm to 5 microns. The wells may be, in an embodiment, 20 nm to 5 microns across (e.g., measured as a distance between neighboring interstitials 362). The wells 360 can be sized to accommodate only one bead 330. Therefore, the size of each well 360, in a specific embodiment, may be no more than twice the bead diameter.

In the workflow if FIG. 12, a silicon dioxide surface with a resin overlayer is provided and patterend via nanoimprinting. The patterned resin is activated with plasma (e.g., O₂ plasma) and treated with a first silence. The treated surface is than provided with a coating, such as a polymethyl methacrylate (PMMA) coating. Oxygen etching exposes a top surface of the underlying patterned resin and removes the first silane from the interstitials 362 while the frist silane is retained in the side and bottom surfaces of the wells 360. A second antifouling treatment is provided to the exposed interstitials, and the PMMA is removed to expose the wells 360. The disclosed workflow is by way of example to generate the substrate 360. The substrate 360 can be used to load beads 330 and used in conjunction with the disclosed techniques.

In some embodiments, as shown in FIG. 13, a plurality of analyte molecules are simultaneously detected in a sample through multiplexing, wherein different pluralities of supramolecular structures 10 provide a plurality of signals (e.g., linker barcode, sample barcode) for sequencing and analyte identification as well as sample demultiplexing. In some embodiments, methods described herein for detecting analytes in a sample provide a high-throughput and high-multiplexing capability by using a plurality of supramolecular structures 10. Each different plurality of supramolecular structures 10a, 10b, 10c have associated different sets of affinity binders 2 that may be a same set between the pluralities or different sets, or different sets. However, in an embodiment, multiplexed analyte detection can be used for different samples that are all contacted with a same set of affinity binders 2 that can all be detected using a same functionalized substrate 60 that has a particular set of capture molecules 70 compatible with the set of affinity binders 2.

The core structures 13 can generally by the same, whereby an individual plurality the core structures 13 are coupled to different affinity binders 2. However, for a particular sample run, all core structures 13 may include or be coupled to one or more sample-specific oligonucleotide barcodes 450a, 450b, 450c that are distinguishable between samples 50. That is, each core structure 13 can include one or more barcodes 450. Thus, the complexes 40a, 40b, 40c can be pooled or run together on the sample substrate, but the detectable signals can be associated with a particular sample based on detection of a particular sample-specific barcode 450.

The multiplexing may include a wash or separation step that separates the complexes 40 from unbound analyte 14. Such separation may include affinity separation via a tag on the supramolecular structure 10, magnetic separation, size separation.

Embodiments of the disclosure include a multiplexing kit, such as an n-plex kit. The kit is provided with universal or common adapters on the core structures 13. The adapter serves as a dock for different n-plex barcodes. The number of barcodes can be chosen based on the number of samples to be multiplexed, and each sample barcode can be added in separate reaction vessels so that the sample-indexed core structures 13 are isolated from one another until tagging, complex formation, and separation from unbound analyte.

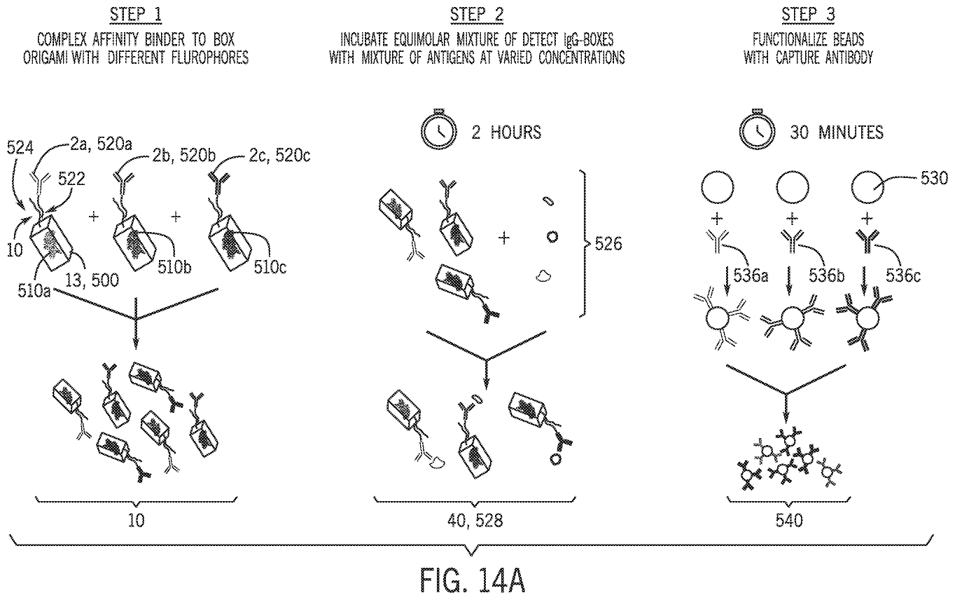

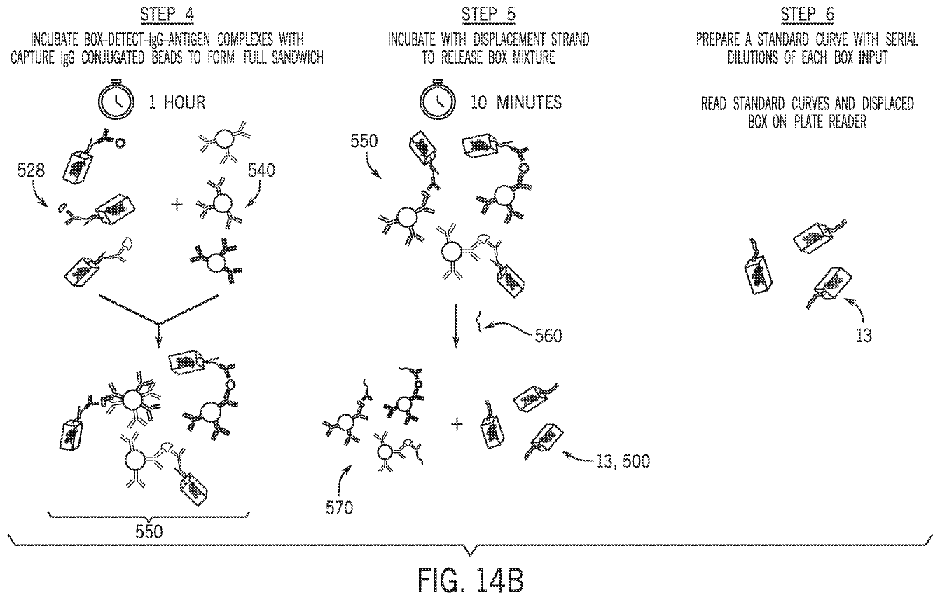

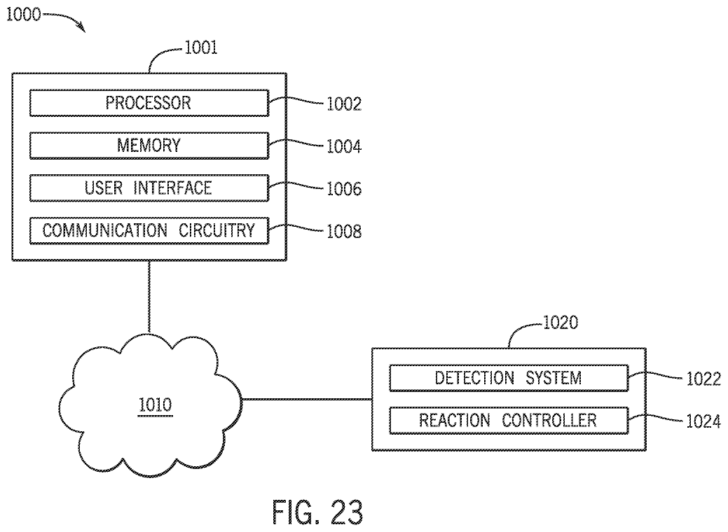

FIG. 14A shows steps of an experimental workflow for antigen detection. The workflow includes functionalizing supramolecular structures 10 to complex with particular affinity binders. For example, the core structure 13 may be implemented as a generally box-shaped core structure 500. However, it should be understood that other shapes and implementations of the core structure 13 are also contemplated. Each core structure 13 is coupled to a signaling element, illustrated here as fluorophores 500. However, the signaling element may be a nucleic acid signal generator, an optical signal generator, an electrical signal generator, a magnetic signal generator. The signaling element may be detected by the detection system as generally discussed with respect to FIG. 23.

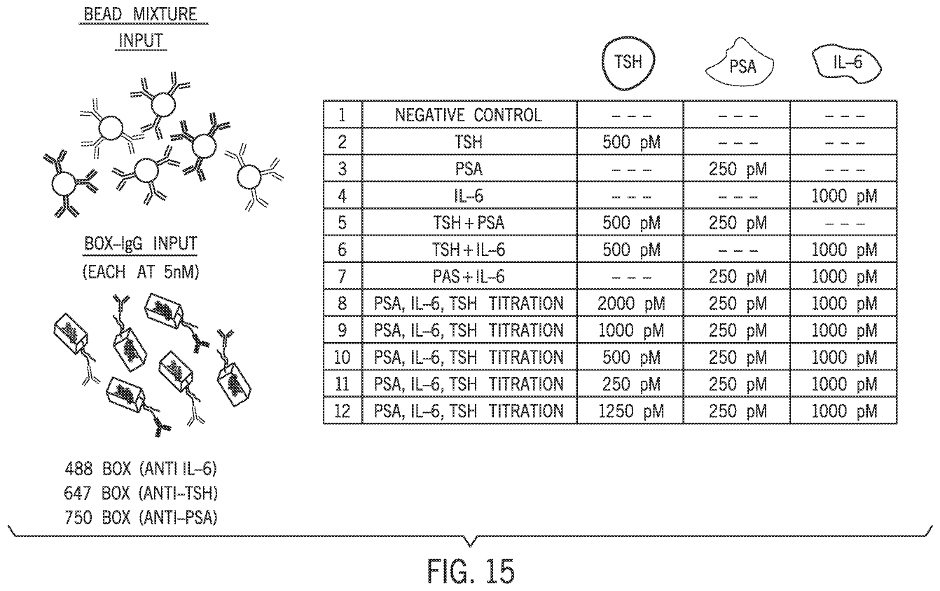

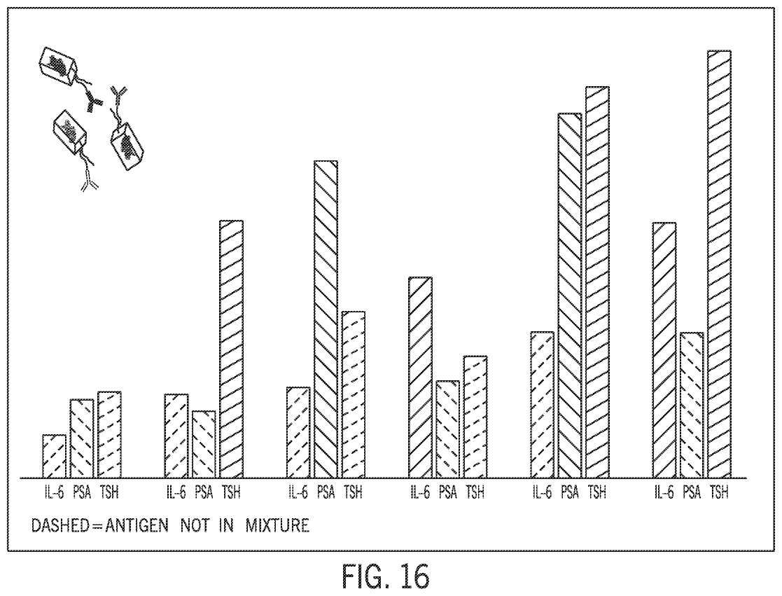

In the experiments discussed with respect to FIGS. 15-16, the affinity binder 2 was IgG antibody 520 coupled to a single-stranded capture strand 520 complexed to a core structure 13 having the box origami structure 500 (e.g., a box structure formed from nucleic acid strands) with multiple associates fluorophores 510 supramolecular structure according to embodiments of the disclosure. However, it should be understood that other antibody types or other affinity binders 2 are also encompassed in the disclosed embodiments. In the illustrated example, each different IgG represents a different affinity binder 2 having respective different specificities for different analytes, e.g., antigens 522. Thus, the illustrated workflow is shown with a threeplex detection capability, and each specific IgG antibody is coupled to a supramolecular structure having a particular fluorophore type. Thus, the fluorophores 510a coupled to the first IgG antibody 520a can be detected at a first fluorescence range, while fluorophores 510b coupled to the second IgG antibody 520b can be detected at a distinguishable second fluorescence range, and fluorophores 510c coupled to the third IgG antibody 520c can be detected at a distinguishable third fluorescence range. In an embodiment, each box origami structure 500 is generally the same other than differing by different fluorescence detection wavelengths depending on the different coupled fluorophores 510a, 510b, 510c. However, in embodiments, the pool of core structures 13 or box origami structures 500 may be the same or different for a particular workflow.

At step one, single-stranded nucleic acid capture strands 522 conjugated to IgG are complexed with a single-stranded complementary strand 524 coupled to the box origami structure 500 to form a duplex structure. Thus, the IgG antibodies are complexed with the core structures 13 via complementary binding under conditions that promote duplex structure formation. Each different antibody 520 and box origami structure 500 may have respective universal capture strands 522 and complementary strands 524 that are the same even for different affinity binding specificity to simplify and batch certain reagent preparation steps. However, in embodiments, each specific IgG antibody 520a, 520b, 520c, and so on, may be coupled to a unique nucleic acid capture strand 522 that may include barcode or other identifying information unique to the particular affinity binder 2.

While the experimental workflow is shown as a threeplex reaction with three different IgG antibodies 520a, 520b, 520c, permitting detection of three different antigens, single, duplex, or other multiplex arrangements are also contemplated. In an embodiment, expanded fluorophore signal resolution can be achieved by controlling a number of fluorphores 510 coupled to each box origami structure 500 to achieve a range of different signal intensities. Thus, affinity binders 2 can be distinguished based on a detected fluorescence range and/or a detected intensity.

At step 2 of the workflow, the pool of supra molecular structures is contacted with a sample that may or may not include antigens 526 of interest. If the antigens 526 to which the IgG antibodies specifically bind are present, assemblies 40 are formed that are a complex 528 of a supramolecular structure 10 bound to the antigen 526.

At step 3 of the workflow, which may be performed before, during, or after steps 1 and 2, beads 530, e.g., magnetic beads, are functionalized with capture antibodies 536 to form functionalized capture beads 540. In one example, the beads 530 may be coupled to streptavidin that binds to biotin-labeled antibodies 536. the capture antibodies 536a, 536b, 536c may have different binding specificity based on the specific binding of the IgG antibodies 520a, 520b, 520c.

FIG. 14B shows additional steps of the experimental workflow of FIG. 14A. At step 4, the supramolecular structure-antigen complexes 528 are contacted with the functionalized capture beads 540 to form sandwich structures 550. For example, the IgG antibodies 520 and the capture antibodies 536 both bind to a particular antigen 526. In an embodiment, the IgG antibody 520a and the capture antibody 536a both bind to an individual antigen 526. Any box origamis 500 without a bound antigen 526 would not be coupled to a magnetic bead 530 and therefore can be removed from the sandwich structures 550 via a magnetic pulldown.

At step 5, a displacing strand 560 is provided into the reaction mix to disrupt the duplex of the capture strand 520 and the complementary strand 522. In an embodiment, the displacement is via toehold-mediated displacement. Thus, in one example, the capture strand 522 includes a complementary region that binds to the complementary strand 522 and a toehold region that is noncomplementary and that remains unbound or un annealed before contact with a displacing strand 560. The displacing strand is complementary to both the complementary region and the toehold region, thus facilitating displacement from the complementary strand 522 via binding of the displacing strand 560 to the toehold region to form a duplex of the displacing strand 560 and the capture strand 520. Accordingly, the displacing strand 560 shares sequence identity with the complementary strand 522 and also include a toehold complement region. Contact with the displacing strand causes the capture strand 520 and the displacing strand 560 to form a duplex that releases the box origami 500 into solution, with the capture strand 520 in a duplex with the displacing strand 560. In an embodiment, the released box origami 500 can be separated from the remaining portion 570 of the sandwich structures 550. For example, the remaining portion 570 retains the beads 530, which can be magnetically pulled down to leave the box origami in solution.

It should be understood that the displacement may be reversed, with the complementary strand 522 can including the toehold region. The displacing strand 560 binds to the complementary strand 522 to disrupt the duplex and to release the box origami into solution, with the capture strand 520 being single-stranded.

At step 6, the released box origamis 500 are imaged at wavelength corresponding to the coupled fluorophores 510, and detectable signal is indicative of a presence of a particular antigen associated with a particular wavelength range. The signal can be assessed for an expected or appropriate optical intensity indicative of antigen presence. For example, the optical intensity can be However, other detection modalities are also contemplated as discussed herein.

In an embodiment, time-separated cycling of different displacement strands 560 with specificity to different capture strands 520 can be used to increase a plexity of the workflow. For example, in the three fluorophore example, N number of sets of three fluorophores can be coupled to box origamis 500 have a set-specific unique capture strand 520 that is displaced by a unique set-specific displacing strand 56. For N number of sets, there can be N number of unique capture strand 520-displacing strand 560 pairs. Each unique displacing strand 560 of a known sequence can be added separately to release only some of the box origamis 500 carrying the corresponding capture strand 520. The released box origamis 500 are imaged to acquire antigen-related data, and the next displacing strand 560 can be added.

FIG. 15 shows different experimental subgroups assessed using the experimental workflow of FIGS. 14A-B. The antigens used were TSH, PSA, and IL-6, and these corresponded to the threeplex differential antibody binding and detection. The different antigens were tests alone and in combination with one another to identity potential cross-binding or other interference. Antibodies were loaded with % antibody loading: 1.5% (Dynabeads) In the experiment, the incubation times were:

30 minutes for Biotinylated-IgG with bead incubation
10 minutes with D-biotin to occupy any free avidin sites

1 hours with box-IgG and antigen

1 hour with Box-IgG-antigen and IgG-Bead 1× PBS pH 7.4 +0.1% tween+10 mM Mg. The displacing strand was 1 uM displacing strand in PBS Tween+10 mM Mg. The different fluorophores were as follows:
IL-6 readout with alexafluor 488 Box
TSH readout with alexafluor 647 Box
PSA readout with alexafluor 750 Box
Detection was via a Tecan microplate reader. It should be understood that the experimental workflow may be conducted with other, more, and/or fewer fluorophores.

FIG. 16 shows experimental results with different antigen combinations of the three antigens used in the experiment of FIG. 15 and showing optical intensity readout. Each of the individual antigens can be detected independently or in a mixture and without significant cross-talk or interference.

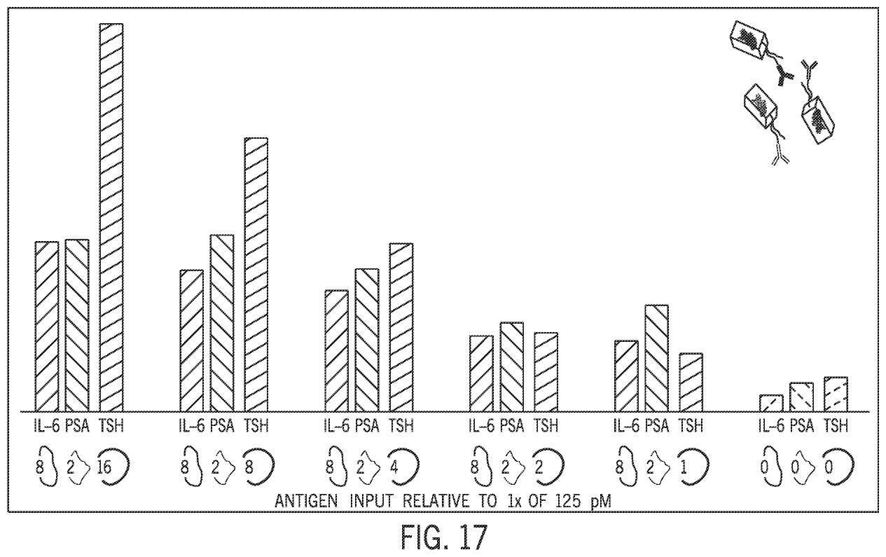

FIG. 17 shows experimental results with different antigen combinations of the three antigens used in the experiment of FIG. 15 and in which TSH is titrated but PSA and IL-6 are held constant. Changing concentration of TSH in the sample is reflected in the detected optical output.

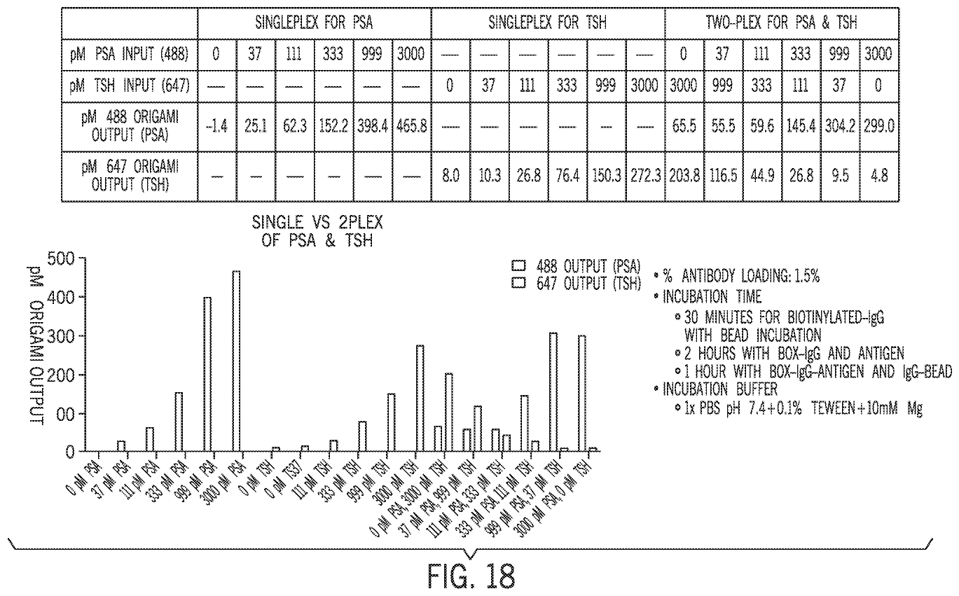

FIG. 18 shows experimental results of an experiment in with titration of TSH or PSA in a duplex reaction and showing that changing concentration of antigen are detectable in the optical output.

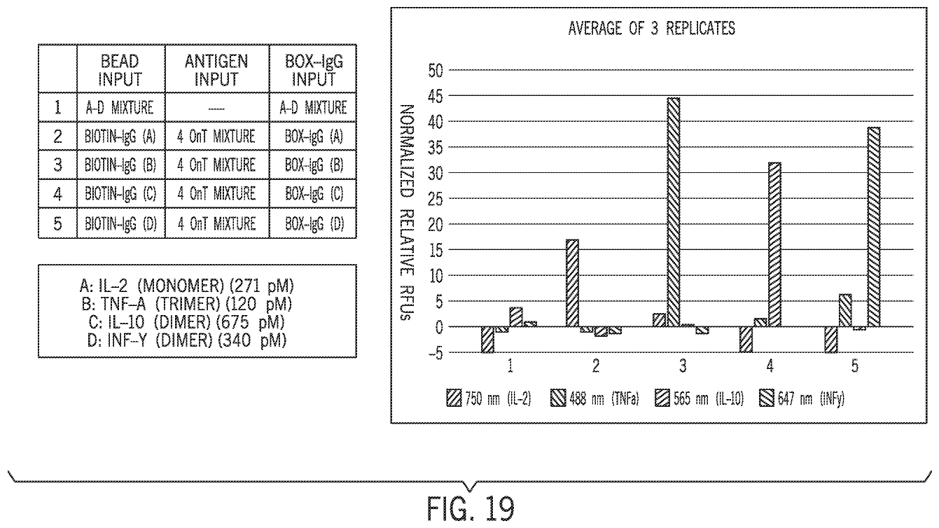

FIG. 19 shows results of a fourplex experiment to quantify four cytokines out of a mixture.

TNFa: readout with alexafluor 488 box (128 imagers)
IL-10: readout with atto 565 box (128 imagers)
INFy: readout with alexafluor 647 box (128 imagers)
IL-2: readout with alexafluor 750 box (128 imagers)
Box origmai IgG inputs all at 5 nM
2 hour incubation with antigen and Box-IgG in 10 mM MOPS+150 mM NaCl+10 mM MgCl₂+0.1% tween-20

1 hour incubation with Box-IgG:antigen+IgG-bead

Displacement of box with 500 nM in 10 mM MOPS+150 mM NaCl+10 mM MgCl₂+0.1% tween-20
Readout on tecan, 15 uL/sample, 140 gain for 491, 550, 641 nm excitation wavelengths
2 out of the 3 replicated were diluted 100× Dimer/trimer antigens have larger readouts.

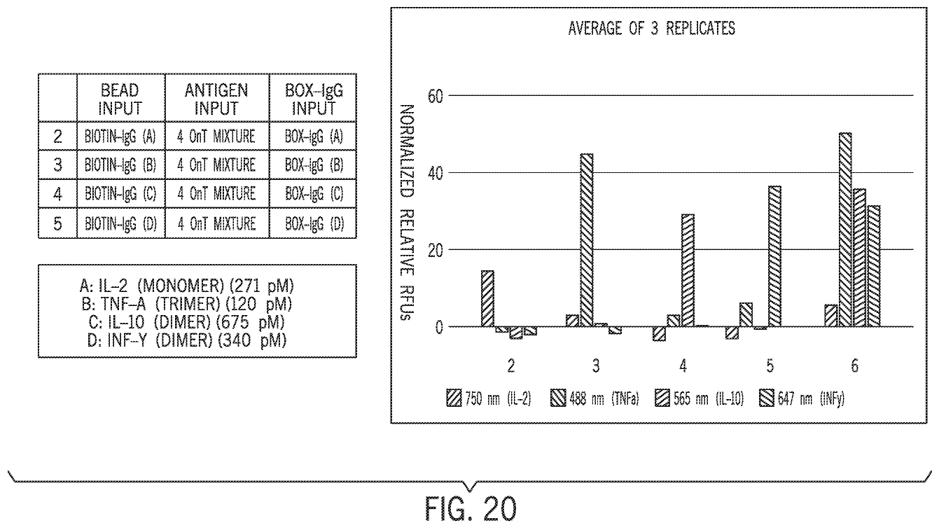

FIG. 20 shows results of an experiment to quantify four cytokines out of a mixture in which only one cytokine per subgroup is detected.

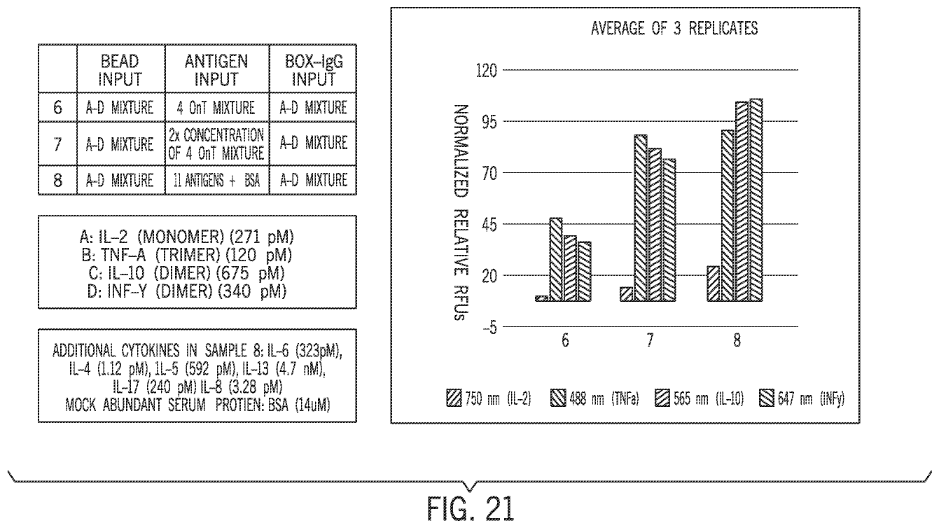

FIG. 21 shows an experiment in which IL-2, TNFα, IL-10, and IFNγ are detected in a mock serum mixture showing that optical detection is seen for antigens in a complex biological sample.

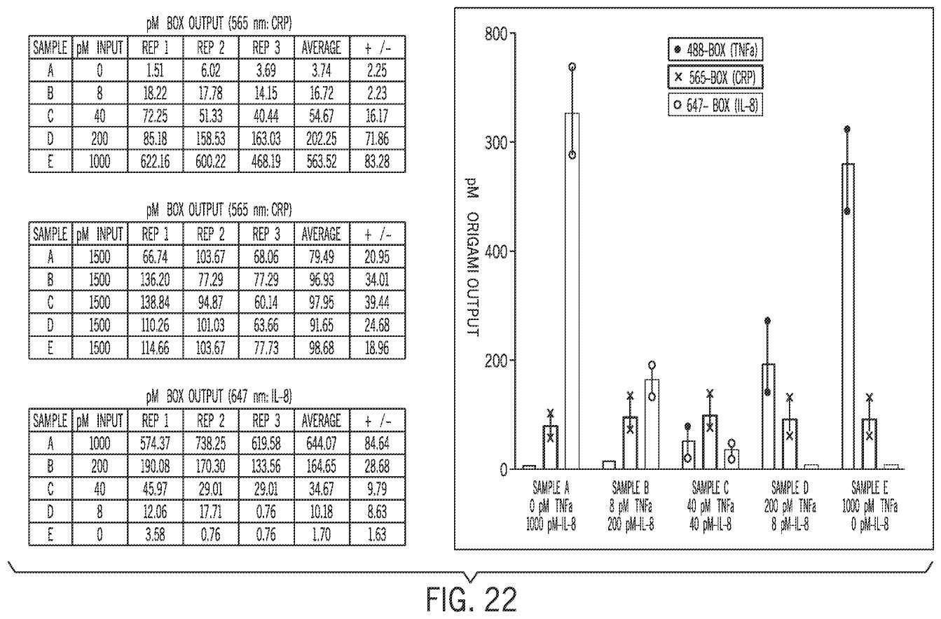

FIG. 22 shows a titration series of IL-8 and TNF alpha, with a constant CRP concentration showing that antigen concentration differences are detectable in the optical output.

The experimental results demonstrated effective solution-based detection for proposed experimental workflows.

Embodiments of the present disclosure include one or more computer-implemented detection systems configured to perform certain methods of the disclosed embodiments. FIG. 23 shows an analyte detection system 1000 that includes a controller 1001. The controller 1001 includes processor 1002 and a memory 1004 storing instructions configured to be executed by the processor 1002. The controller 1001 includes a user interface 1006 and communication circuitry 1008, e.g., to facilitate communication over the internet 1010 and/or over a wireless or wired network. The user interface 1006 facilitates user interaction with characterized analyte detection results as provided herein.

The processor 1002 is programmed to receive analyte detection data and characterize the detected analytes. In one embodiment, the processor generates a report of detected analytes in a sample after incubation of supramolecular structure complexes 40 with an array of binding sites 66 and detection of features of the binding site 66, the complexes 10, or both. The report may include data of generated optical signals at various binding sites that corresponds to a detected analyte binding event. The report may include processed data, such as a list of detected analytes or positive/negative binding results. The report may include a list of available affinity binders of complexes 10 and/or available capture molecules 70 that is indicative of the analyte detection capabilities.

The system 1000 also includes an analyte detector 1020 that operates to detect analyte binding via detection of one or more components of the supramolecular structure 10 (and/or any immobilized supramolecular structures 160 of the binding sites 66). The analyte detector 1020 includes a detection system having one or more sensors 1022. The analyte detector 1020 may also include a reaction controller 1024 that controls sample incubation and appropriate release of reaction reagents and detector molecule assemblies at appropriate time points. The sensor 1022 may be one or more of an optical sensor (e.g., a fluorescent sensor, an infrared sensor), an image sensor, an electrical sensor, or a magnetic sensor. In an embodiment, the sensor 102 is a metal-oxide semiconductor image sensor device.

The supramolecular structures 10 of complexes 40 retained at individual binding sites 66 via interaction with capture molecules 70 are detected to generate a detectable signal. For example, the barcode of the linker 20 is used as a binding site for a detectable signaling element (e.g., via hybridization of a complementary sequence) that is contacted with the bound complexes. In some embodiments, the signaling element comprises a fluorescent molecule or microbead, a fluorescent polymer, highly charged nanoparticles or polymer. In some embodiments, the barcode is amplified. For example, the barcode is used as a polymerization initiator for growth of highly fluorescent polymer in a process such as rolling circle amplification or hybridization chain reaction.

In some embodiments, the signaling element is optically active and can be measured using a microscope or integrated optically sensor within the substrate 60. In some embodiments, the signaling element is electrically active and may be measured using an integrated electrical sensor. In some embodiments, the signaling element is magnetically active and may be measured using an integrated magnetic sensor. In some embodiments, each signal event is associated with the capture of the same type of analyte molecule (a single copy of the same type of analyte molecule), determined by the corresponding detector and affinity binder, thus counting the number of locations where the signaling element is present gives the quantification of the analyte molecule in the sample

In some embodiments, the supramolecular structure converts information about the presence of a given analyte molecule in a sample to a DNA signal. In some embodiments, the DNA signal corresponds to sequence data for a capture barcode and/or detector barcode, wherein the affinity binder and detector molecule are simultaneously linked to (e.g., bound to) the analyte molecule (e.g., sandwich formation).

In some embodiments, detecting the presence of an analyte molecule, as described herein, comprises controllably releasing a single, or multiple, unique nucleic acid molecules into the solution to be used to identify as well as quantify properties of the analyte molecule from the sample. In some embodiments, said unique nucleic acid molecules are provided by capture barcodes 20 of the respective supramolecular structures. In some embodiments, detecting the presence of an analyte molecule, as described herein, comprises creating an optical or electrical signal connected to the state change that can be counted to quantify the concentration of the analyte molecule in solution.

In some embodiments, each supramolecular structure 10 comprises unique DNA barcodes corresponding to the associated affinity binder. As provided herein, the linker barcode 20 can be used to uniquely identify individual supramolecular structures 10. In turn, each supramolecular structure 10 is assembled so that the affinity binder 2 may be associated with the capture barcode 20, e.g., stored in a lookup table of an analyte detection system. Thus, when the capture barcode 20 is identified, the identity of the affinity binder 2 is also accessible.

The analyte detection technique that can be used to characterize analyte binding. The bound complexes 40 can be characterized based on 1) the barcode 20 of the supramolecular structure 10 and, in embodiments, 2) a barcode of the capture molecule 70 that is co-located with the barcode 20. The barcodes can be detected by a signal generated by a detector molecule coupled to one or more of the binding sites 66, the complexes 40, or the capture molecules 70. In an embodiment, the locations of the capture molecules 70 can be determined before complex binding. That is, the array of binding sites 66 may be provided pre-mapped, or the mapping can be a separate step. The mapping may include a step of detecting the capture molecule barcodes as generally provided herein, such as detecting a unique optical, electrical, and/or magnetic pattern. In an embodiment, the detection includes sequencing a nucleotide sequence of the capture barcode. In an embodiment, the detection includes amplification and quantitation of the amplified product, e.g., detection of a signal associated with a probe via qPCR.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for detecting an analyte molecule present in a sample, the method comprising:
providing a sample comprising analyte molecules;
contacting the sample with a pool of supramolecular structures in solution to form analyte molecule-supramolecular structure complexes, an individual analyte molecule-supramolecular structure complex comprising:
a core structure comprising a plurality of core molecules;
an affinity binder linked to the core structure; and
an analyte molecule of the analyte molecules bound to the affinity binder,
wherein different supramolecular structures of the pool of supramolecular structures comprise different affinity binders with different binding affinity for other analyte molecules of the analyte molecules;
contacting the analyte molecule-supramolecular structure complexes with an array,
wherein binding sites of the array comprise respective immobilized affinity binders with binding affinity for different analyte molecules; and
detecting binding of the analyte molecule-supramolecular structure complexes to binding sites of the array.

2. The method of claim 1, comprising identifying the individual analyte molecule of the individual analyte molecule-supramolecular structure bound to the individual binding site of the array.

3. The method of claim 2, wherein the identifying comprises: generating a detectable signal from the supramolecular structure and associating the detectable signal with the individual binding site.

4. The method of claim 3, wherein the detectable signal is an optical, magnetic, or electrical signal indicative of a presence of the individual analyte molecule-supramolecular structure at the individual binding site.

5. The method of claim 2, wherein the identifying comprises: amplifying an initiator of the supramolecular structure and detecting the amplifying.

6. The method of claim 5, wherein the initiator is a nucleic acid.

7. The method of claim 2, wherein the identifying comprises: amplifying an initiator immobilized on the individual binding site and detecting the amplifying.

8. The method of claim 7, wherein the initiator is a nucleic acid.

9. The method of claim 1, wherein the affinity binder and the immobilized affinity binders are antibody molecules or portions of antibody molecules.

10. The method of claim 1, wherein binding of the analyte molecule-supramolecular structure to an immobilized affinity binders at an individual binding site comprising binding of the affinity binding of the supramolecular structure to a first portion of the analyte molecule and binding of the immobilized affinity binder to second portion of the analyte molecule.

11. The method of claim 1, wherein the immobilized affinity binders are linked to each individual binding site via nucleic acid capture molecules.

12. The method of claim 11, wherein each individual binding site comprises a plurality of nucleic acid capture molecules.

13. The method of claim 12, wherein the plurality of nucleic acid capture molecules at an individual binding site all have a same nucleic acid sequence.

14. The method of claim 12, wherein the plurality of nucleic acid capture molecules are coupled to an immobilized supramolecular structure.

15. The method of claim 1, wherein each individual binding site comprises a plurality of immobilized affinity binders all having affinity for a same analyte molecule of the analyte molecules.

16. The method of claim 1, wherein each binding site has a diameter between 20-500 nanometers.

17. The method of claim 1, wherein each supramolecular structure of the pool is a nanostructure.

18. The method of claim 14, wherein each core structure is a nanostructure.

19. The method of claim 1, wherein each supramolecular structure of the pool is arranged into a pre-defined shape and/or have a prescribed molecular weight.

20. The method of claim 1, further comprising removing analyte molecule-supramolecular structures not bound to binding sites of the array after contacting the sample with the array.

21.-36 (canceled)

37. An array for analyte detection, comprising:
a plurality of binding sites patterned on a substrate and separated by interstitial surfaces of the substrate, wherein each binding site comprises a well, and wherein at least one surface of the well comprises a first chemical group and the interstitial surfaces comprise a second chemical group;
a bead loaded into each of the binding sites, wherein the first chemical group selectively binds to the bead and the second chemical group does not interact with or bind to the bead, and wherein each bead comprises:
a plurality of single-stranded oligonucleotides immobilized on each bead such that an individual binding site of the array comprises a plurality of single-stranded oligonucleotides comprising a same sequence that is distinguishable from sequences of other single-stranded oligonucleotides immobilized on different binding sites of the array;
affinity binders captured at binding sites of the array such that the captured affinity binders form capture molecules immobilized at respective binding sites; and
analyte molecules bound to individual capture molecules at the individual binding site, wherein each analyte molecule is complexed with a supramolecular structure detection assembly.

38. A method for detecting an analyte molecule present in a sample, the method comprising:
providing a plurality of supramolecular structures, wherein each supramolecular structure comprises
a core structure comprising a plurality of core molecules;
an antibody having binding affinity for an antigen and coupled to a nucleic acid capture strand; and
a complementary strand to the nucleic acid capture strand, wherein the complementary strand is linked to the core structure and forms a duplex structure with the nucleic acid capture strand to couple the antibody to the core structure, wherein the plurality of supramolecular structures comprise different antibodies relative to one another with respective different binding affinities;
contacting the plurality of supramolecular structures with a sample comprising the antigen such that the antigen binds to the antibody to form a complex;
contacting the complex with a bead carrying a capture antibody having binding specificity for the antigen to form a sandwich structure;
displacing the nucleic acid capture strand from the complementary strand using a displacing strand to release the core structure into solution, wherein the released core structure is not coupled to the antibody; and
detecting the core structure.

39. The method of claim 38, wherein the core structure is a box origami formed from nucleic acids.

40. The method of claim 39, wherein the box origami is coupled to a plurality of fluorophores that fluoresce at a particular wavelength, and wherein detecting the core structure comprises detecting fluorescence at the particular wavelength.

41. The method of claim 38, wherein the capture strand comprises a toehold region that does not bind to the complementary strand but that does bind to the displacing strand.

42. The method of claim 38, wherein the complementary strand comprises a toehold region that does not bind to the capture strand but that does bind to the displacing strand.

43. The method of claim 38, comprising separating a remaining portion of the sandwich structure from the released core structure before the detecting.

44. (canceled)
